SEQUENCE LISTING (24 sequences)

SEQ ID NO: 1 - protein, 14 aa, Artificial Sequence
  Synthetic construct
TGTGSDVGSYNLVS

SEQ ID NO: 2 - protein, 7 aa, Artificial Sequence
  Synthetic construct
GDSERPS

SEQ ID NO: 3 - protein, 10 aa, Artificial Sequence
  Synthetic construct
SSYAGSGIYV

SEQ ID NO: 4 - protein, 5 aa, Artificial Sequence
  Synthetic construct
TYAMG

SEQ ID NO: 5 - protein, 5 aa, Artificial Sequence
  Synthetic construct
DYAMG

SEQ ID NO: 6 - protein, 5 aa, Artificial Sequence
  Synthetic construct
NYAMG

SEQ ID NO: 7 - protein, 14 aa, Artificial Sequence
  Synthetic construct
SIGSSGAQTRYADS

SEQ ID NO: 8 - protein, 14 aa, Artificial Sequence
  Synthetic construct
SIGASGSQTRYADS

SEQ ID NO: 9 - protein, 14 aa, Artificial Sequence
  Synthetic construct
SIGASGAQTRYADS

SEQ ID NO: 10 - protein, 14 aa, Artificial Sequence
  Synthetic construct
SIGASGGQTRYADS

SEQ ID NO: 11 - protein, 7 aa, Artificial Sequence
  Synthetic construct
LAIGDSY

SEQ ID NO: 12 - protein, 14 aa, Artificial Sequence
  Synthetic construct
  MISC_FEATURE (1)..(1): Xaa is a polar or hydrophobic amino acid, e.g. Thr, Ala, Ser, or Ile
  MISC_FEATURE (12)..(12): Xaa is a hydrophobic amino acid, e.g. Leu or Ile
XGTGSDVGSYNXVS

SEQ ID NO: 13 - protein, 7 aa, Artificial Sequence
  Synthetic construct
  MISC_FEATURE (3)..(3): Xaa is a polar amino acid, e.g. Ser, Asn, or Thr
  MISC_FEATURE (4)..(4): Xaa is a polar or acidic amino acid, e.g. Gln, Glu, or Asn
GDXXRPS

SEQ ID NO: 14 - protein, 10 aa, Artificial Sequence
  Synthetic construct
  MISC_FEATURE (1)..(1): Xaa is a polar or hydrophobic amino acid, e.g. Cys, Ser, Ile, or Tyr
  MISC_FEATURE (4)..(4): Xaa is a hydrophobic amino acid, e.g. Ala or Val
XSYXGSGIYV

SEQ ID NO: 15 - protein, 5 aa, Artificial Sequence
  Synthetic Construct
  MISC_FEATURE (1)..(1): Xaa is a polar or acidic amino acid, e.g. Glu, Thr, Asp, or Asn
XYAMG

SEQ ID NO: 16 - protein, 14 aa, Artificial Sequence
  Synthetic Construct
  MISC_FEATURE (4)..(4): Xaa is a polar or hydrophobic amino acid, e.g. Ser or Ala
  MISC_FEATURE (7)..(7): Xaa is Gly, Ser, or Ala
  MISC_FEATURE (10)..(10): Xaa is a basic amino acid, e.g. Gly, Ser, or Ala
SIGXSGXQTXYADS

SEQ ID NO: 17 - protein, 7 aa, Artificial Sequence
  Synthetic Construct
  MISC_FEATURE (3)..(3): Xaa is a hydrophobic or basic amino acid, e.g. Ile, Leu, or His
  MISC_FEATURE (4)..(4): Xaa is Gly, Ser, Asp, Gln, or His
LAXXDSY

SEQ ID NO: 18 - protein, 14 aa, Artificial Sequence
  Synthetic Construct
  MISC_FEATURE (4)..(4): Xaa is a polar or hydrophobic amino acid, e.g. Ser or Ala
  MISC_FEATURE (7)..(7): Xaa is Glu, Ser, or Ala
SIGXSGXQTRYADS

SEQ ID NO: 19 - protein, 216 aa, Artificial Sequence
  Synthetic Construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 20 - protein, 445 aa, Artificial Sequence
  Synthetic Construct
EVQLLESGGGLVQPGGSLRLSCAASGFTFSTYAMGWVRQAPGKGLEWVSSIGSSGAQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 445 aa, Artificial Sequence
  Synthetic Construct
EVQLLESGGGLVQPGGSLRLSCAASGFTFSDYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 445 aa, Artificial Sequence
  Synthetic Construct
EVQLLESGGGLVQPGGSLRLSCAASGFTFSNYAMGWVRQAPGKGLEWVSSIGASGAQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 445 aa, Artificial Sequence
  Synthetic Construct
EVQLLESGGGLVQPGGSLRLSCAASGFTFSTYAMGWVRQAPGKGLEWVSSIGASGGQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 445 aa, Artificial Sequence
  Synthetic Construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2022/0259308 A1
(43) Pub. Date: Aug. 18, 2022

(54) FCRN ANTIBODIES AND METHODS OF USE THEREOF

(71) Applicant: JANSSEN BIOTECH, INC., Horsham, PA (US)

(72) Inventors: Santiago ARROYO, Boston, MA (US); William DENNEY, Watertown, MA (US)

(21) Appl. No.: 17/597,928

(22) PCT Filed: Aug. 3, 2020
(86) PCT No.: PCT/US2020/044731

§ 371 Date: Jan. 28, 2022

Related U.S. Application Data
(60) Provisional application No. 62/881,897, filed on Aug. 1, 2019.

(51) Int. Cl. C07K 16/28; A61P 37/06

(52) U.S. Cl. CPC C07K 16/283; A61K 2039/505; A61P 37/06

(57) ABSTRACT

Methods for intravenous dosing of antibodies to human neonatal Fc receptor (FcRn) are described. The anti-FcRn antibodies are useful, e.g., to promote clearance of autoantibodies in a subject, to suppress antigen presentation in a subject, to block an immune response, e.g., block an immune complex-based activation of the immune response in a subject, or to treat immunological diseases (e.g., autoimmune diseases) in a subject.

### CLAIM OF PRIORITY

This application claims the benefit of U.S. Provisional Patent Application Ser. No. 62/881,897, filed Aug. 1, 2019. The entire contents of the foregoing are hereby incorporated by reference.

### BACKGROUND

Therapeutic proteins, e.g., therapeutic antibodies, have rapidly become a clinically important drug class for patients with immunological diseases. Numerous autoimmune and alloimmune diseases are mediated by pathogenic antibodies. There exists a need for novel methods of treating immunological diseases.

### SUMMARY

The present disclosure features methods for intravenous dosing of antibodies to human neonatal Fc receptor (FcRn). The anti-FcRn antibodies are useful, e.g., to promote clearance of autoantibodies in a subject, to suppress antigen presentation in a subject, to block an immune response, e.g., block an immune complex-based activation of the immune response in a subject, or to treat immunological diseases (e.g., autoimmune diseases) in a subject.

Described herein is method of treating an alloimmune and/or autoimmune disorder, comprising intravenous infusion of a 5-60 or 30-60 mg/kg dose of an anti-FcRn antibody to a subject, wherein the intravenous infusion takes place over 90 minutes or less and wherein the anti-FcRn antibody comprises: (1) a light chain variable region comprising a CDR L1, a CDR L2, and a CDR L3 and (2) a heavy chain variable region comprising a CDR H1, a CDR H2, and a CDR H3, wherein: the CDR L1 comprises a sequence having no more than two amino acid substitutions relative to the sequence of TGTGSDVGSYNLVS (SEQ ID NO: 1), the CDR L2 comprises a sequence having no more than one amino acid substitutions relative to the sequence of GDSERPS (SEQ ID NO: 2), the CDR L3 comprises a sequence having no more than one amino acid substitutions relative to the sequence of SSYAGSGIYV (SEQ ID NO: 3), the CDR H1 comprises a sequence having no more than one amino acid substitutions relative to the sequence of TYAMG (SEQ ID NO: 4), DYAMG (SEQ ID NO: 5), or NYAMG (SEQ ID NO: 6), the CDR H2 comprises a sequence having no more than two amino acid substitutions relative to the sequence of SIGSSGAQTRYADS (SEQ ID NO: 7), SIGASGSQTRYADS (SEQ ID NO: 8), SIGASGAQTRYADS (SEQ ID NO: 9), or SIGASGGQTRYADS (SEQ ID NO: 10), and the CDR H3 comprises a sequence having no more than one amino acid substitutions relative to the sequence of LAIGDSY (SEQ ID NO: 11).

In various embodiments: the CDR L1 comprises the sequence TGTGSDVGSYNLVS (SEQ ID NO: 1), the CDR L2 comprises the sequence GDSERPS (SEQ ID NO: 2), the CDR L3 comprises the sequence SSYAGSGIYV (SEQ ID NO: 3), the CDR H1 comprises the sequence TYAMG (SEQ ID NO: 4), the CDR H2 comprises the sequence SIGASGSQTRYADS (SEQ ID NO: 8), and the CDR H3 comprises the sequence LAIGDSY (SEQ ID NO: 11); infusion takes place over 7-90 minutes, 7-60 minutes, 7-45 minutes, 7-30 minutes, 10-90 minutes, 10-60 minutes, 10-45 minutes, 10-30 minutes or 15-30 minutes; the Fc domain of the antibody is not fucosylated; the Fc domain of the antibody is not glycosylated; the alloimmune and/or autoimmune disorder is selected from the group consisting of fetal and neonatal alloimmune thrombocytopenia, hemolytic disease of the fetus and newborn, alloimmune pan-thrombocytopenia, congenital heart block, fetal arthrogryposis, neonatal myasthenia gravis, neonatal autoimmune hemolytic anemia, neonatal anti-phospholipid syndrome, neonatal polymyositis, dermatomyositis, neonatal lupus, neonatal scleroderma, Behcet's disease, neonatal Graves' disease, neonatal Kawasaki disease, neonatal autoimmune thyroid disease, and neonatal type I diabetes mellitus.

In various embodiments: the alloimmune and/or autoimmune disorder is selected from the group consisting of thrombocytopenia, pan-thrombocytopenia, congenital heart block, arthrogryposis, myasthenia gravis, autoimmune hemolytic anemia, warm autoimmune hemolytic anemia, anti-phospholipid syndrome, polymyositis, dermatomyositis, lupus, scleroderma, Behcet's disease, Graves' disease, Kawasaki disease, autoimmune thyroid disease, and type I diabetes mellitus.

In various embodiments: the infusion is infusion of a composition comprising 5-60 mg/ml of the antibody; the infusion is infusion of a composition comprising 30 mg/ml of the antibody; the heavy chain comprises a sequence having at least 95%, 97%, 99%, or 100% identity to the sequence of any one of SEQ ID NOs: 20-24 and the light chain comprises a sequence having at least 95%, 97%, 99%, or 100% identity to the sequence of SEQ ID NO: 19; the antibody heavy chain comprises the amino acid sequence of any of SEQ ID Nos: 20-24 with amino acid other than N at position 296 of SEQ ID NOs: 20-24; the infusion is infusion of a composition comprising 10-60 mg/ml of the antibody, 20-30 mM sodium phosphate, 20-30 mM sodium chloride, 80-100, and 0.1-0.005% w/v Polysorbate 80; the antibody heavy chain comprises the amino acid sequence of SEQ ID NO:24 with one or more of the following amino acid substitutions: A23V, S30R, L80V, A84T, E85D, A93V, relative to the sequence of SEQ ID NO: 24 and the antibody light chain comprises the amino acid sequence of SEQ ID NO: 19 with one or more of the following amino acid substitutions: Q38H, V58I, and G99D, relative to the sequence of SEQ ID NO: 19; the antibody heavy chain does not contain a C-terminal lysine; the administered antibody comprises a light chain comprising SEQ ID NO: 19 and a heavy chain comprising SEQ ID NO:24 or a variant of SEQ ID NO:24 wherein the amino acid at 296 is other than N; the antibody is administered at 5-30 mg/kg; the concentration of antibody in the intravenous infusion is between 10 mg/ml and 30 mg/ml.

In various embodiments: the subject is a pregnant woman; the dose is based on the weight of the pregnant woman at first dosing and is not adjusted upward based on weight gain by the pregnant woman; the dose is dose per administration and is based on the weight of the pregnant woman at first dosing and is adjusted upward based on weight gain by the pregnant woman; the dose is administered at least every other week; the dose is administered every other week; the dose is administered at least every week; the dose is administered every week; the subject is a pregnant woman and the first dose is administered during the first trimester of pregnancy; the subject is a pregnant woman and the first dose is administered during the second trimester of pregnancy; the subject is a pregnant woman and the first dose is administered during the third trimester of pregnancy; the subject is a pregnant woman and the pregnant woman has an obstetrical history of severe fetal anemia; the subject is a pregnant woman and the pregnant woman has an obstetrical history of hemolytic disease of the fetus and newborn; the subject is a pregnant woman and the pregnant woman has an elevated anti RhD, anti-Rhc or anti Kell immunoglobulin alloantibody titer; the subject is a pregnant woman and the pregnant woman has an elevated anti-Rhc or anti-Kell immunoglobulin alloantibody titer; the subject is a pregnant woman and the pregnant woman has an elevated immunoglobulin alloantibody titer for one or more antibodies selected from the group consisting of anti-Lua, Lub, Bg, Kna, Yta, E. c. K. Cw, Fya, cE, ce, D, Ce, cE, K, Kpa, Kpb, Fya, M, N, S, Lea, Leb, Fy, Jka. Diego, P and Mia/Mur; the subject is a pregnant woman and the pregnant woman has an obstetrical history of severe fetal anemia or stillbirth at ≤24 weeks gestation and elevated anti-D or anti-Kell IgG alloantibody titers and is pregnant with an antigen-positive fetus; the subject is a pregnant woman and the first dosing is weeks 12 to 16 of pregnancy; and the subject is a pregnant woman and the first dosing is during week 14 of pregnancy.

In one aspect, the isolated antibody contains: (1) a light chain variable region that includes a CDR L1, a CDR L2, and a CDR L3 and (2) a heavy chain variable region that includes a CDR H1, a CDR H2, and a CDR H3, wherein the CDR L1 comprises a sequence having no more than two amino acid substitutions relative to the sequence of TGTGSDVGSYNLVS (SEQ ID NO: 1), the CDR L2 comprises a sequence having no more than one amino acid substitutions relative to the sequence of GDSERPS (SEQ ID NO: 2), the CDR L3 comprises a sequence having no more than one amino acid substitutions relative to the sequence of SSYAGSGIYV (SEQ ID NO: 3), the CDR H1 comprises a sequence having no more than one amino acid substitutions relative to the sequence of TYAMG (SEQ ID NO: 4), DYAMG (SEQ ID NO: 5), or NYAMG (SEQ ID NO: 6), the CDR H2 comprises a sequence having no more than two amino acid substitutions relative to the sequence of SIGSSGAQTRYADS (SEQ ID NO: 7), SIGASGSQTRYADS (SEQ ID NO: 8), SIGASGAQTRYADS (SEQ ID NO: 9), or SIGASGGQTRYADS (SEQ ID NO: 10), and the CDR H3 comprises a sequence having no more than one amino acid substitutions relative to the sequence of LAIGDSY (SEQ ID NO: 11).

In some embodiments, the antibody binds human FcRn with a K_{D} of less than 200, 150, 100, 50, or 40 pM.

In some embodiments, the isolated antibody contains a CDR L1 having the sequence of TGTGSDVGSYNLVS (SEQ ID NO: 1), a CDR L2 having the sequence of GDSERPS (SEQ ID NO: 2), a CDR L3 having the sequence of SSYAGSGIYV (SEQ ID NO: 3), a CDR H1 having the sequence of TYAMG (SEQ ID NO: 4), a CDR H2 having the sequence of SIGSSGAQTRYADS (SEQ ID NO: 7), and a CDR H3 having the sequence of LAIGDSY (SEQ ID NO: 11).

In some embodiments, the isolated antibody contains a CDR L1 having the sequence of TGTGSDVGSYNLVS (SEQ ID NO: 1), a CDR L2 having the sequence of GDSERPS (SEQ ID NO: 2), a CDR L3 having the sequence of SSYAGSGIYV (SEQ ID NO: 3), a CDR H1 having the sequence of DYAMG (SEQ ID NO: 5), a CDR H2 having the sequence of SIGASGSQTRYADS (SEQ ID NO: 8), and a CDR H3 having the sequence of LAIGDSY (SEQ ID NO: 11).

In some embodiments, the isolated antibody contains a CDR L1 having the sequence of TGTGSDVGSYNLVS (SEQ ID NO: 1), a CDR L2 having the sequence of GDSERPS (SEQ ID NO: 2), a CDR L3 having the sequence of SSYAGSGIYV (SEQ ID NO: 3), a CDR H1 having the sequence of NYAMG (SEQ ID NO: 6), a CDR H2 having the sequence of SIGASGAQTRYADS (SEQ ID NO: 9), and a CDR H3 having the sequence of LAIGDSY (SEQ ID NO: 11).

In other embodiments, the isolated antibody contains a CDR L1 having the sequence of TGTGSDVGSYNLVS (SEQ ID NO: 1), a CDR L2 having the sequence of GDSERPS (SEQ ID NO: 2), a CDR L3 having the sequence of SSYAGSGIYV (SEQ ID NO: 3), a CDR H1 having the sequence of TYAMG (SEQ ID NO: 4), a CDR H2 having the sequence of SIGASGGQTRYADS (SEQ ID NO: 10), and a CDR H3 having the sequence of LAIGDSY (SEQ ID NO: 11).

In yet other embodiments, the isolated antibody contains a CDR L1 having the sequence of TGTGSDVGSYNLVS (SEQ ID NO: 1), a CDR L2 having the sequence of GDSERPS (SEQ ID NO: 2), a CDR L3 having the sequence of SSYAGSGIYV (SEQ ID NO: 3), a CDR H1 having the sequence of TYAMG (SEQ ID NO: 4), a CDR H2 having the sequence of SIGASGSQTRYADS (SEQ ID NO: 8), and a CDR H3 having the sequence of LAIGDSY (SEQ ID NO: 11).

In some embodiments, the light chain of the isolated antibody comprises a sequence having at least 90% identity to the sequence of

(SEQ ID NO: 19)
QSALTQPASVSGSPGQSITISCTGTGSDVGSYNLVSWYQ
QHPGKAPKLMIYGDSERPSGVSNRFSGSKSGNTASLTIS
GLQAEDEADYYCSSYAGSGIYVFGTGTKVTVLGQPKAAP
SVTLFPPSSEELQANKATLVCLISDFYPGAVTVAWKADS
SPVKAGVETTTPSKQSNNKYAASSYLSLTPEQWKSHKSY
SCQVTHEGSTVEKTVAPTECS.

In some embodiments, the heavy chain of the isolated antibody comprises a sequence having at least 90% identity to the sequence of

(SEQ ID NO: 20)
EVQLLESGGGLVQPGGSLRLSCAASGFTFSTYAMGWVRQAPGKGLEWV
SSIGSSGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In other embodiments, the heavy chain of the isolated antibody comprises a sequence having at least 90% identity to the sequence of

(SEQ ID NO: 21)
EVQLLESGGGLVQPGGSLRLSCAASGFTFSDYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVICVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In other embodiments, the heavy chain of the isolated antibody comprises a sequence having at least 90% identity to the sequence of

(SEQ ID NO: 22)
EVQLLESGGGLVQPGGSLRLSCAASGFTFSNYAMGWVRQAPGKGLEWV
SSIGASGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDILMISRTPEVICVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the heavy chain of the isolated antibody comprises a sequence having at least 90% identity to the sequence of

(SEQ ID NO: 23)
EVQLLESGGGLVQPGGSLRLSCAASGFTFSTYAMGWVRQAPGKGLEWV
SSIGASGGQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVICVVVDVSHEDPEVKFNVVYVDGVEVHNAK
TKPREEQYASTYRVVSVLTVLHQDWLNGKEYKCKVSNKALPAPIEKTI
SKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEAL
HNHYTQKSLSLSPG.

In other embodiments, the heavy chain of the isolated antibody comprises a sequence having at least 90% identity to the sequence of

(SEQ ID NO: 24)
EVQLLESGGGLVQPGGSLRLSCAASGFTFSTYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDILMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In another aspect, the isolated antibody has a light chain and a heavy chain, wherein the light chain comprises a sequence having at least 90%, 95%, 98% or 99% identity to the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises a sequence having at least 90%, 95%, 98% or 99% identity to the sequence of

(SEQ ID NO: 20)
EVQLLESGGGLVQPGGSLRLSCAASGFTFSTYAMGWVRQAPGKGLEWV
SSIGSSGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In another aspect, the isolated antibody containing has light chain and a heavy chain, wherein the light chain comprises a sequence having at least 90%, 95%, 98% or 99% identity to the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises a sequence having at least 90%, 95%, 98% or 99% identity to the sequence of

(SEQ ID NO: 21)
EVQLLESGGGLVQPGGSLRLSCAASGFTFSDYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDILMISRTPEVICVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In another aspect, the isolated antibody has a light chain and a heavy chain, wherein the light chain comprises a sequence having at least 90%, 95%, 98% or 99% identity to the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises a sequence having at least 90%, 95%, 98% or 99% identity to the sequence of

(SEQ ID NO: 22)
EVQLLESGGGLVQPGGSLRLSCAASGFTFSNYAMGWVRQAPGKGLEWV
SSIGASGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDILMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In another aspect, the isolated antibody has a light chain and a heavy chain, wherein the light chain comprises a sequence having at least 90%, 95%, 98% or 99% identity to the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises a sequence having at least 90%, 95%, 98% or 99% identity to the sequence of

(SEQ ID NO: 23)
EVQLLESGGGLVQPGGSLRLSCAASGFTFSTYAMGWVRQAPGKGLEWV
SSIGASGGQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDILMISRTPEVICVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In yet another aspect, the isolated antibody has a light chain and a heavy chain, wherein the light chain comprises a sequence having at least 90%, 95%, 98% or 99% identity to the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises a sequence having at least 90%, 95%, 98% or 99% identity to the sequence of

(SEQ ID NO: 24)
EVQLLESGGGLVQPGGSLRLSCAASGFTFSTYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In some embodiments, the antibody comprising a light chain variable region comprising an amino acid sequence that is at least 95%, 97%, 99%, or 100% identical to: QSALTQPASVSGSPGQSITISCTGTGSDVGSYNLVSWYQQHPGKAPKLMIYGDSERPSGVSNR FSGSKSGNTASLTISGLQAEDEADYYCSSYAGSGIYVFGTGTKVTVL (SEQ ID NO: X). In some embodiments, the light chain variable region contains a CDR L1 having the sequence of TGTGSDVGSYNLVS (SEQ ID NO: 1), a CDR L2 having the sequence of GDSERPS (SEQ ID NO: 2), a CDR L3 having the sequence of SSYAGSGIYV (SEQ ID NO: 3). In some embodiments, the antibody comprising a heavy chain variable region comprising an amino acid sequence that is at least 95%, 97%, 99%, or 100% identical to: EVQLLESGGGLVQPGGSLRLSCAASGFTFSTYAMGWVRQAPGKGLEWVSSIGASGSQTRYADS VKGRFTISRDNSKNTLYLQMNSLRAEDTAVYYCARLAIGDSYWGQGTMVTVSS (SEQ ID NO: Y). In some embodiments, the heavy chain variable region contains a CDR H1 having the sequence of TYAMG (SEQ ID NO: 4), a CDR H2 having the sequence of SIGASGSQTRYADS (SEQ ID NO: 8), and a CDR H3 having the sequence of LAIGDSY (SEQ ID NO: 11).

In some embodiments, the heavy chain of the isolated antibody comprises a sequence having at least 95%, 97%, 99%, or 100% identity to the sequence of any one of SEQ ID NOs: 20-24. In other embodiments, the light chain of the isolated antibody comprises a sequence having at least 95%, 97%, 99%, or 100% identity to the sequence of SEQ ID NO: 19.

In some embodiments, the heavy chain of the isolated antibody comprises a sequence having no more than 5, 4, 3, 2 or 1 single amino acid substitutions relative to the amino acid sequence of any one of SEQ ID NOs: 20-24. In some embodiments, the light chain of the isolated antibody comprises a sequence having no more than 5, 4, 3, 2 or 1 single amino acid substitutions relative to the sequence of SEQ ID NO: 19.

In some embodiments, the isolated antibody further includes amino acid substitution N297A, relative to the sequence of any one of SEQ ID NOs: 20-24 (According to EU Numbering).

In other embodiments, the isolated antibody further includes amino acid substitutions D355E and L357M, relative to the sequence of any one of SEQ ID NOs: 20-24. (According to EU Numbering).

In other embodiments, the isolated antibody further includes any one or more of the following amino acid substitutions: A23V, S30R, L80V, A84T, E85D, A93V (According to EU Numbering), relative to the sequence of any one of SEQ ID NOs: 20-24 and Q38H, V58I, and G99D (According to EU Numbering), relative to the sequence of SEQ ID NO: 19.

In yet other embodiment, the isolated antibody does not contain a C-terminal lysine at residue 446, relative to the sequence of any one of SEQ ID NOs: 20-24.

In some embodiments, the antibody of any of the above aspects binds human FcRn with a K_{D} that is less than or equal to that of an antibody having the light chain variable region and heavy chain variable region of N022, N023, N024, N026, or N027 and also having the same Fc region as that of the antibody being compared. For example, in a particular K_{D} assay, the K_{D} of the antibody is less than 200, 150, 100, 50, or 40 pM.

The amino acid positions assigned to complementary determining regions (CDRs) and framework regions (FRs) of any isolated antibody described herein are defined according to EU index of Kabat (Sequences of Proteins of Immunological Interest, 5th Ed. Public Health Service, National Institutes of Health, Bethesda, Md. (1991)).Fc region sequence positions are according to EU numbering (Edelman et al., Proc Natl Acad USA, 63:78-85 (1969).

In another aspect, the isolated antibody has a light chain and a heavy chain, wherein the light chain comprises or consists of the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises or consists of the sequence of

(SEQ ID NO: 20)
EVQLLESGGGLVQPGGSLRLSCAASGFTFSTYAMGWVRQAPGKGLEWV
SSIGSSGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In another aspect, the isolated antibody has a light chain and a heavy chain, wherein the light chain comprises or consists of the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises or consists of the sequence of

(SEQ ID NO: 21)
EVQLLESGGGLVQPGGSLRLSCAASGFTFSDYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDILMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In another aspect, the isolated antibody has a light chain and a heavy chain, wherein the light chain comprises the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises or consists of the sequence of

(SEQ ID NO: 22)
EVQLLESGGGLVQPGGSLRLSCAASGFTFSNYAMGWVRQAPGKGLEWV
SSIGASGAQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In another aspect, the isolated antibody has a light chain and a heavy chain, wherein the light chain comprises or consists of the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises or consists of the sequence of

(SEQ ID NO: 23)
EVQLLESGGGLVQPGGSLRLSCAASGFTFSTYAMGWVRQAPGKGLEWV
SSIGASGGQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In yet another aspect, the isolated antibody has a light chain and a heavy chain, wherein the light chain comprises or consists of the sequence of QSALTQPASVSGSPGQSITISCTGTGSDVGSYNLVSWYQQHPGKAPKLMIYGDSERPSGVSNRFSGSKSGN TASLTISGLQAEDEADYYCSSYAGSGIYVFGTGTKVTVLGQPKAAPSVTLFPPSSEELQANKATLVCLISDFYP GAVTVAWKADSSPVKAGVETTTPSKQSNNKYAASSYLSLTPEQWKSHKSYSCQVTHEGSTVEKTVAPTEC S (SEQ ID NO: 19); and the heavy chain comprises or consists of the sequence of

(SEQ ID NO: 24)
EVQLLESGGGLVQPGGSLRLSCAASGFTFSTYAMGWVRQAPGKGLEWV
SSIGASGSQTRYADSVKGRFTISRDNSKNTLYLQMNSLRAEDTAVYYC
ARLAIGDSYWGQGTMVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYASTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

In another aspect, the disclosure features a method of increasing IgG catabolism in a subject. In another aspect, the disclosure features a method of reducing autoantibodies in a subject. In yet another aspect, the disclosure features a method of treating or reducing an immune complex-based activation of an immune response in a subject. The methods include administering to the subject any isolated antibody described herein or a pharmaceutical composition including any isolated antibody described herein.

In some embodiments, the immune response in the subject is an acute or chronic immune response.

In some embodiments, the subject has or the acute immune response is activated by a medical condition selected from the group consisting of pemphigus vulgaris, lupus nephritis, myasthenia gravis, Guillain-Barre syndrome, antibody-mediated rejection, catastrophic anti-phospholipid antibody syndrome, immune complex-mediated vasculitis, glomerulitis, a channelopathy, neuromyelitis optica, autoimmune hearing loss, idiopathic thrombocytopenia purpura (ITP), autoimmune haemolytic anaemia (AIHA), immune neutropenia, dilated cardiomyopathy, and serum sickness.

In some embodiments, the subject has or the chronic immune response is activated by a medical condition selected from the group consisting of chronic inflammatory demyelinating polyneuropathy (CIDP), systemic lupus, a chronic form of a disorder indicated for acute treatment, reactive arthropathies, primary biliary cirrhosis, ulcerative colitis, and antineutrophil cytoplasmic antibody (ANCA)-associated vasculitis.

In some embodiments, the subject has or the immune response is activated by an autoimmune disease. In particular, the autoimmune disease is selected from the group consisting of alopecia areata, ankylosing spondylitis, antiphospholipid syndrome, Addison's disease, hemolytic anemia, autoimmune hepatitis, hepatitis, Behcet's disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, and Wegener's granulomatosis.

In another aspect, the disclosure features a method of treating a fetal and neonatal alloimmune and/or autoimmune disorder comprising, consisting of, or consisting essentially of IV administration of an antibody described herein to a pregnant subject

In some embodiments of all aspects, the subject has a history of having had a previous fetal and neonatal alloimmune and/or autoimmune disorder. For example, in some embodiments the pregnant subject has previously had a pregnancy wherein the fetus or neonate has had a fetal and neonatal alloimmune and/or autoimmune disorder. In some embodiments of all aspects, the subject is at risk of having a fetal and neonatal alloimmune and/or autoimmune disorder.

In some embodiments of all aspects, the fetal and neonatal alloimmune and/or autoimmune disorder is selected from the group consisting of fetal and neonatal alloimmune thrombocytopenia, hemolytic disease of the fetus and newborn, alloimmune pan-thrombocytopenia, congenital heart block, fetal arthrogryposis, neonatal myasthenia gravis, neonatal autoimmune hemolytic anemia, neonatal anti-phospholipid syndrome, neonatal polymyositis, dermatomyositis, neonatal lupus, neonatal scleroderma, Behcet's disease, neonatal Graves' disease, neonatal Kawasaki disease, neonatal autoimmune thyroid disease, and neonatal type I diabetes mellitus. In some embodiments of all aspects, the fetal and neonatal autoimmune and/or autoimmune disorder is hemolytic disease of the fetus and newborn. In some embodiments of all aspects, the fetal and neonatal autoimmune and/or autoimmune disorder is fetal and neonatal alloimmune thrombocytopenia. In some embodiments of all aspects, the fetal and neonatal autoimmune and/or autoimmune disorder is congenital heart block.

In some embodiments of all aspects, treatment reduces the risk of a miscarriage.

In some embodiments of all aspects, the subject has a history of having had a previous fetal and neonatal alloimmune and/or autoimmune disorder. For example, in some embodiments, the pregnant subject has had a previous pregnancy wherein the fetus or neonate had a fetal and neonatal alloimmune and/or autoimmune disorder. In some embodiments of all aspects, the subject is at risk of having a fetal and neonatal alloimmune and/or autoimmune disorder.

In some embodiments of all aspects, the fetal and neonatal alloimmune and/or autoimmune disorder is selected from the group consisting of fetal and neonatal alloimmune thrombocytopenia, hemolytic disease of the fetus and newborn, alloimmune pan-thrombocytopenia, congenital heart block, fetal arthrogryposis, neonatal myasthenia gravis, neonatal autoimmune hemolytic anemia, neonatal anti-phospholipid syndrome, neonatal polymyositis, dermatomyositis, neonatal lupus, neonatal scleroderma, Behcet's disease, neonatal Graves' disease, neonatal Kawasaki disease, neonatal autoimmune thyroid disease, and neonatal type I diabetes mellitus. In some embodiments of all aspects, the fetal and neonatal autoimmune and/or autoimmune disorder is hemolytic disease of the fetus and newborn. In some embodiments of all aspects, the fetal and neonatal autoimmune and/or autoimmune disorder is fetal and neonatal alloimmune thrombocytopenia. In some embodiments of all aspects, the fetal and neonatal autoimmune and/or autoimmune disorder is congenital heart block. In some embodiments of all aspects, treatment reduces the risk of a miscarriage.

In some embodiments of all aspects, the method treats the pregnant subject, a fetus of the pregnant subject, and/or a combination thereof.

In some embodiments of all aspects, the autoimmune disorder is selected from the group consisting of alopecia areata, ankylosing spondylitis, antiphospholipid syndrome, Addison's disease, hemolytic anemia, autoimmune hepatitis, hepatitis, Behcet's disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, or Wegener's granulomatosis.

Also described is a method of reducing the risk of or reducing the risk of developing an autoimmune or alloimmune disorder, comprising, consisting of, or consisting essentially of IV administration of an FcRn antibody described herein to a pregnant subject

In another aspect, the disclosure features a method of increasing antibody catabolism in a subject, the method comprising, consisting of, or consisting essentially of IV administration of an antibody described herein to a pregnant subject.

In some embodiments of all aspects, increasing antibody catabolism comprises increasing pathogenic antibody catabolism. In some embodiments of all aspects, the pathogenic antibody is pathogenic to the mother, the fetus, or both the mother and the fetus. In some embodiments of all aspects, the pathogenic antibody is an IgG antibody. In some embodiments of all aspects, the antibody causes a fetal and neonatal alloimmune and/or autoimmune disorder in a fetus in the pregnant subject.

In some embodiments of all aspects, the fetal and neonatal alloimmune and/or autoimmune disorder is selected from the group consisting of fetal and neonatal alloimmune thrombocytopenia hemolytic disease of the fetus and newborn, alloimmune pan-thrombocytopenia, congenital heart block, fetal arthrogryposis, neonatal myasthenia gravis, neonatal autoimmune hemolytic anemia, neonatal anti-phospholipid syndrome, neonatal polymyositis, dermatomyositis, neonatal lupus, neonatal scleroderma, Behcet's disease, neonatal Graves' disease, neonatal Kawasaki disease, neonatal autoimmune thyroid disease, and neonatal type I diabetes mellitus.

In another aspect, the disclosure features a method of reducing autoantibodies in a subject, the method comprising, consisting of, or consisting essentially of administering an antibody described herein to a pregnant subject.

In some embodiments of all aspects, the immune response is an acute or chronic immune response in the subject.

In some embodiments of all aspects, the acute immune response is activated by a medical condition selected from the group consisting of pemphigus vulgaris, lupus nephritis, myasthenia gravis, Guillain-Barre syndrome, antibody-mediated rejection, catastrophic anti-phospholipid antibody syndrome, immune complex-mediated vasculitis, glomerulitis, a channelopathy, neuromyelitis optica, autoimmune hearing loss, idiopathic thrombocytopenia purpura, autoimmune haemolytic anaemia, immune neutropenia, dilated cardiomyopathy, and serum sickness. For example, in some embodiments, the acute immune response is activated by a medical condition in the pregnant subject. For example, in some embodiments, the acute immune response is activated in the fetus or neonate by a medical condition in the pregnant subject. In some embodiments of all aspects, the acute immune response is activated by a medical condition in the pregnant subject. In some embodiments of all aspects, the acute immune response is activated in the fetus or neonate by a medical condition in the pregnant subject. In some embodiments of all aspects, the acute immune response is activated by idiopathic thrombocytopenia purpura. In some embodiments of all aspects, the acute immune response is activated by pemphigus vulgaris. In some embodiments of all aspects, the acute immune response is activated by catastrophic anti-phospholipid antibody syndrome. In some embodiments of all aspects, the acute immune response is activated by neuromyelitis optica. In some embodiments of all aspects, the acute immune response is activated by antibody-mediated rejection. In some embodiments of all aspects, the acute immune response is activated by myasthenia gravis.

Also described herein is a method of treating a fetal and neonatal alloimmune and/or autoimmune disorder comprising, consisting of, or consisting essentially of administering M281, e.g., an antibody having the light chain sequence of SEQ ID NO:19 and the heavy chain sequence of SEQ ID NO:24 (or a variant thereof (e.g., a variant in which the amino acid at position 296 of SEQ ID NO:24 is other than N), e.g., at a dose of 15 mg/kg or 30 mg/kg (e.g., a weekly dose), to a subject, e.g., a pregnant subject. In some cases the method includes ceasing administration if the subject exhibits hypoalbuminemia (e.g., a serum albumin level below 30 g/l, 25 g/l, 20 g/l). Also described is method comprising, consisting of, or consisting essentially of treating a fetal and neonatal alloimmune and/or autoimmune disorder comprising administering an antibody described herein to a pregnant subject (e.g., at a dose of 15 mg/kg or 30 mg/kg, e.g., a weekly dose) and administering albumin if the subject exhibits hypoalbuminemia (e.g., a serum albumin level below 30 g/l, 25 g/l, 20 g/l). Also described is method comprising, consisting of, or consisting essentially of treating a fetal and neonatal alloimmune and/or autoimmune disorder comprising administering an antibody described herein to a pregnant subject (e.g., at a dose of 15 mg/kg or 30 mg/kg, e.g., a weekly dose) and administering a hyperosmolar solution (e.g., mannitol or other solution known in the art) if the subject exhibits hypoalbuminemia (e.g., a serum albumin level below 30 g/l, 25 g/l, 20 g/l). Also described is a method comprising, consisting of, or consisting essentially of treating a fetal and neonatal alloimmune and/or autoimmune disorder comprising administering M281 (e.g., at a dose of 15 mg/kg or 30 mg/kg, e.g., a weekly dose) to a pregnant subject and testing the serum albumin level of the subject at least once prior to or subsequent to administration of M281. In some cases of this method, administration of M281 can be continued or not.

In some embodiments of all aspects, the chronic immune response is activated by a medical condition selected from the group consisting of chronic inflammatory demyelinating polyneuropathy (CIDP), systemic lupus, reactive arthropathies, primary biliary cirrhosis, ulcerative colitis, and antineutrophil cytoplasmic antibody-associated vasculitis. In some embodiments of all aspects, the chronic immune response is activated by chronic inflammatory demyelinating polyneuropathy.

In some embodiments of all aspects, the subject has an autoimmune disease. In some embodiments of all aspects, the autoimmune disease is selected from the group consisting of alopecia areata, ankylosing spondylitis, antiphospholipid syndrome, Addison's disease, hemolytic anemia, warm autoimmune hemolytic anemia, anti-factor antibodies, heparin induced thrombocytopenia (, sensitized transplant, autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, and Wegener's granulomatosis. In some embodiments of all aspects, the autoimmune disease is warm autoimmune hemolytic anemia. In some embodiments of all aspects, the autoimmune disease is anti-factor antibodies. In some embodiments of all aspects, the autoimmune disease heparin induced thrombocytopenia. In some embodiments of all aspects, the autoimmune disease is sensitized transplant.

In another aspect, the disclosure features a method of decreasing antibody transport across the placenta of a pregnant subject, the method comprising, consisting of,

In another aspect, the disclosure features a method of treating an antibody-mediated enhancement of viral disease in a fetus or a neonate, the method comprising, consisting of, or consisting essentially of administering an antibody to a pregnant subject, wherein the antibody comprises, consists of, or consists essentially of: a light chain and a heavy chain, wherein the light chain comprises, consists of, or consists essentially of a sequence having at least 90% identity to the sequence of SEQ ID NO: 19; and the heavy chain comprises, consists of, or consists essentially of a sequence having at least 90% identity to the sequence selected from the group consisting of SEQ ID NO: 20, SEQ ID NO: 21, SEQ ID NO: 22, SEQ ID NO: 23, and SEQ ID NO: 24.

In another aspect, the disclosure features a method of treating an antibody-mediated enhancement of viral disease in a fetus or a neonate, the method comprising, consisting of, or consisting essentially of administering an antibody to a pregnant subject, wherein the antibody comprises, consists of, or consists essentially of: a light chain and a heavy chain, wherein the light chain comprises, consists of, or consists essentially of the sequence of SEQ ID NO: 19; and the heavy chain comprises, consists of, or consists essentially of the sequence selected from the group consisting of SEQ ID NO: 20, SEQ ID NO: 21, SEQ ID NO: 22, SEQ ID NO: 23, and SEQ ID NO: 24.

In some embodiments of all aspects, the viral disease is caused by a virus selected from the group consisting of an alpha virus infection, flavivirus infection, Zika virus infection, Chikungunya virus infection, Ross River virus infection, severe acute respiratory syndrome coronavirus infection, Middle East respiratory syndrome, avian influenza infection, influenza virus infection, human respiratory syncytial virus infection, Ebola virus infection, yellow fever virus infection, dengue virus infection, human immunodeficiency virus infection, respiratory syncytial virus infection, Hantavirus infection, Getah virus infection, Sindbis virus infection, Bunyamwera virus infection, West Nile virus infection, Japanese encephalitis virus B infection, rabbitpox virus infection, lactate dehydrogenase elevating virus infection, reovirus infection, rabies virus infection, foot-and-mouth disease virus infection, porcine reproductive and respiratory syndrome virus infection, simian hemorrhagic fever virus infection, equine infectious anemia virus infection, caprine arthritis virus infection, African swine fever virus infection, lentivirus infection, BK papovavirus infection, Murray Valley encephalitis virus infection, enterovirus infection, cytomegalovirus infection, pneumovirus infection, morbillivirus infection, and measles virus infection.

In some embodiments of all aspects, the pregnant subject has or is at risk of having a medical condition that activates an immune response in the pregnant subject. In some embodiments of all aspects, the medical condition is pemphigus vulgaris, lupus nephritis, myasthenia gravis, Guillain-Barre syndrome, antibody-mediated rejection, catastrophic anti-phospholipid antibody syndrome, immune complex-mediated vasculitis, glomerulitis, a channelopathy, neuromyelitis optica, autoimmune hearing loss, idiopathic thrombocytopenia purpura, autoimmune haemolytic anaemia, immune neutropenia, dialated cardiomyopathy, serum sickness, chronic inflammatory demyelinating polyneuropathy, systemic lupus, reactive arthropathies, primary biliary cirrhosis, ulcerative colitis, antineutrophil cytoplasmic antibody (ANCA)-associated vasculitis, alopecia areata, ankylosing spondylitis, antiphospholipid syndrome, Addison's disease, hemolytic anemia, autoimmune hepatitis, hepatitis, Behcet's disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, and Wegener's granulomatosis.

In some embodiments of all aspects, the pregnant subject has a history of having had a previous fetus or neonate that had a fetal and neonatal alloimmune and/or autoimmune disorder. For example, in some embodiments, the pregnant subject has had a previous pregnancy, wherein the fetus or neonate had a fetal and neonatal alloimmune and/or autoimmune disorder.

In some embodiments of all aspects, an antibody associated with an immune disease is detected in a biological sample obtained from the pregnant subject. In some embodiments of all aspects, the biological sample is a blood or urine sample. In some embodiments of all aspects, the biological sample is a blood sample.

In another aspect, the disclosure features a method for treating or reducing the risk of developing a fetal and neonatal alloimmune and/or autoimmune disorder, the method including: IV administration to a pregnant woman of a composition comprising an antibody comprising a light chain having the amino acid sequence of SEQ ID NO:19 and a heavy chain having the amino acid sequence of SEQ ID NO:24 (M281), wherein the administration of M281 ceases after week 34 gestational age.

In another aspect, the disclosure features a method for treating or reducing the risk of developing a fetal and neonatal alloimmune and/or autoimmune disorder comprising administering to a pregnant woman a composition comprising an antibody comprising a light chain having the amino acid sequence of SEQ ID NO:19 and a heavy chain having the amino acid sequence of SEQ ID NO:24 (M281), wherein the administration of M281 ceases at least one week prior to birth.

In various aspects of all methods, the method includes: administering IVIG to the pregnant woman after cessation of administration of M281 and prior to birth (e.g., 40-100 hrs or 1-15 days prior to birth); administration of M281 ceases after gestational week 35; administration of M281 ceases prior to gestational week 36, 37 or 38; the IVIG is administered at 200 mg/kg-1000 mg/kg based on the weight of the pregnant woman; M281 is administered at 30 mg/kg based on the weight of the pregnant woman; M281 is administered at 15 mg/kg based on the weight of the pregnant woman; the dose is dose per administration and is based on the weight of the pregnant woman at first dosing and is not adjusted upward based on weight gain by the pregnant woman; the dose is dose per administration and is based on the weight of the pregnant woman at first dosing and is adjusted upward based on weight gain by the pregnant woman; the composition is administered at least every other week; the composition is administered every other week; the composition is administered at least every week; the composition is administered every week; administration is begun during the first trimester of pregnancy; administration is begun during the second trimester of pregnancy; administration is begun during the third trimester of pregnancy; the route of administration is intravenous; the pregnant woman has an obstetrical history of severe fetal anemia; the pregnant woman has an elevated anti RhD, anti-Rhc or anti Kell immunoglobulin alloantibody titer; the pregnant woman has an elevated anti-Rhc or anti-Kell immunoglobulin alloantibody titer; the pregnant woman has an elevated immunoglobulin alloantibody titer for one or more antibodies selected from the group consisting of anti-Lua, Lub, Bg, Kna, Yta, E. c. K. Cw, Fya, cE, ce, D, Ce, cE, K, Kpa, Kpb, Fya, M, N, S, Lea, Leb, Fy, Jka. Diego, P and Mia/Mur; the pregnant woman has an obstetrical history of severe fetal anemia or stillbirth at 524 weeks gestation and elevated anti-D or anti-Kell IgG alloantibody titers and is pregnant with an antigen-positive fetus; the first dosing is weeks 12 to 16 of pregnancy; the first dosing is during week 14 of pregnancy; and administration is begun during the first trimester of pregnancy.

In various aspects of all methods, the infusion times are identical and takes place over 90 minutes or less, 60 minutes or less, 45 minutes or less, 30 minutes or less, 15 minutes or less, or 7 minutes or less. In various aspects of all methods, the first infusion takes place over 90 minutes or less, 60 minutes or less, 45 minutes or less, 30 minutes or less, 15 minutes or less, and subsequent infusion times are reduced. In some embodiments, the second fusion and the third fusion times are identical, takes place over 60 minutes or less, 45 minutes or less, 30 minutes or less, 15 minutes or less, or 7 minutes or less, and subsequent infusion times are reduced. In various aspects of all methods, the first infusion and the second fusion times are identical, take place over 90 minutes or less, 60 minutes or less, 45 minutes or less, 30 minutes or less, 15 minutes or less, and subsequent infusion times are reduced. In various aspects of all methods, the first infusion takes place over 60 minutes and subsequent infusions take place over 45 minutes or less, 30 minutes or less, or 15 minutes or less; or the first infusion takes place over 45 minutes and subsequent infusions takes place over 30 minutes or less or 15 minutes or less; or the first infusion takes place over 30 minutes and subsequent infusions takes place over 15 minutes or less. In various aspects of all methods, the first infusion and the second fusion both take place over 60 minutes and subsequent infusions take place over 45 minutes or less, 30 minutes or less, or 15 minutes or less; or the first infusion and the second fusion both take place over 45 minutes and subsequent infusions take place over 30 minutes or less or 15 minutes or less; or the first infusion and the second fusion both take place over 30 minutes and subsequent infusions take place over 15 minutes or less.

Described herein, inter alia, is a method of administering an anti-FcRn antibody to a subject comprising intravenous infusion of a 5-60 mg/kg dose of the anti-FcRn antibody to a subject, wherein the intravenous infusion takes place over 90 minutes or less and wherein the anti-FcRn antibody comprises: (1) a light chain variable region comprising a CDR L1, a CDR L2, and a CDR L3 and (2) a heavy chain variable region comprising a CDR H1, a CDR H2, and a CDR H3, wherein

the CDR L1 comprises a sequence having no more than two amino acid substitutions relative to the sequence of TGTGSDVGSYNLVS (SEQ ID NO: 1),

the CDR L2 comprises a sequence having no more than one amino acid substitutions relative to the sequence of GDSERPS (SEQ ID NO: 2),

the CDR L3 comprises a sequence having no more than one amino acid substitutions relative to the sequence of SSYAGSGIYV (SEQ ID NO: 3),

the CDR H1 comprises a sequence having no more than one amino acid substitutions relative to the sequence of TYAMG (SEQ ID NO: 4), DYAMG (SEQ ID NO: 5), or NYAMG (SEQ ID NO: 6),

the CDR H2 comprises a sequence having no more than two amino acid substitutions relative to the sequence of SIGSSGAQTRYADS (SEQ ID NO: 7), SIGASGSQTRYADS (SEQ ID NO: 8), SIGASGAQTRYADS (SEQ ID NO: 9), or SIGASGGQTRYADS (SEQ ID NO: 10), and

the CDR H3 comprises a sequence having no more than one amino acid substitutions relative to the sequence of LAIGDSY (SEQ ID NO: 11).

In some embodiments,

the CDR L1 comprises the sequence TGTGSDVGSYNLVS (SEQ ID NO: 1),
the CDR L2 comprises the sequence GDSERPS (SEQ ID NO: 2),
the CDR L3 comprises the sequence SSYAGSGIYV (SEQ ID NO: 3),
the CDR H1 comprises the sequence TYAMG (SEQ ID NO: 4),
the CDR H2 comprises the sequence SIGASGSQTRYADS (SEQ ID NO: 8), and
the CDR H3 comprises the sequence LAIGDSY (SEQ ID NO: 11).

In some embodiments of all the methods described herein, the infusion takes place over 7-90 minutes, 7-60 minutes, 7-45 minutes, 7-30 minutes, 10-90 minutes, 10-60 minutes, 10-45 minutes, 10-30 minutes or 15-30 minutes.

In some embodiments of all the methods described herein, the Fc domain of the antibody is not fucosylated. In some embodiments of all the methods described herein, the Fc domain of the antibody is not glycosylated. In some embodiments of all the methods described herein, the antibody is an IgG1 antibody. In some embodiments of all the methods described herein, the antibody is a fully human antibody. In some embodiments of all the methods described herein, the subject has a alloimmune and/or autoimmune disorder selected from the group consisting of fetal and neonatal alloimmune thrombocytopenia, hemolytic disease of the fetus and newborn, alloimmune pan-thrombocytopenia, congenital heart block, fetal arthrogryposis, neonatal myasthenia gravis, neonatal autoimmune hemolytic anemia, neonatal anti-phospholipid syndrome, neonatal polymyositis, dermatomyositis, neonatal lupus, neonatal scleroderma, Behcet's disease, neonatal Graves' disease, neonatal Kawasaki disease, neonatal autoimmune thyroid disease, and neonatal type I diabetes mellitus. In some embodiments of all the methods described herein, the subject has a alloimmune and/or autoimmune disorder is selected from the group consisting of thrombocytopenia, pan-thrombocytopenia, congenital heart block, arthrogryposis, myasthenia gravis, autoimmune hemolytic anemia, warm autoimmune hemolytic anemia, anti-phospholipid syndrome, polymyositis, dermatomyositis, lupus, scleroderma, Behcet's disease, Graves' disease, Kawasaki disease, autoimmune thyroid disease, and type I diabetes mellitus.

In some embodiments of all the methods described herein, the infusion is infusion of a composition comprising 5-60 mg/ml of the antibody. In some embodiments of all the methods described herein, the infusion is infusion of a composition comprising 30, 45, or 60 mg/ml of the antibody. In some embodiments of all the methods described herein, the heavy chain comprises a sequence having at least 95%, 97%, 99%, or 100% identity to the sequence of any one of SEQ ID NOs: 20-24 and the light chain comprises a sequence having at least 95%, 97%, 99%, or 100% identity to the sequence of SEQ ID NO: 19. In some embodiments of all the methods described herein, the antibody heavy chain comprises the amino acid sequence of any of SEQ ID Nos: 20-24 with amino acid other than N at position 296 of SEQ ID NOs: 20-24.

In some embodiments of all the methods described herein, the infusion is infusion of a composition comprising 10-60 mg/ml (or 10, 20 or 30 mg/ml) of the antibody, 20-30 mM sodium phosphate, 20-30 mM sodium chloride, 80-100 mg/ml Trehalose, and 0.1-0.005% w/v Polysorbate 80 at pH 6.5.

In some embodiments of all the methods described herein, the antibody heavy chain comprises the amino acid sequence of SEQ ID NO:24 with one or more of the following amino acid substitutions: A23V, S30R, L80V, A84T, E85D, A93V, relative to the sequence of SEQ ID NO: 24 and the antibody light chain comprises the amino acid sequence of SEQ ID NO: 19 with one or more of the following amino acid substitutions: Q38H, V58I, and G99D, relative to the sequence of SEQ ID NO: 19. In some embodiments of all the methods described herein, the antibody heavy chain does not contain a C-terminal lysine. In some embodiments of all the methods described herein, the administered antibody comprises a light chain comprising SEQ ID NO: 19 and a heavy chain comprising SEQ ID NO:24 or a variant of SEQ ID NO:24 wherein the amino acid at 296 is other than N.

In some embodiments of all the methods described herein, the antibody is administered at 5-30 mg/kg. In some embodiments of all the methods described herein, the antibody is administered at 30-60 mg/kg. In some embodiments of all the methods described herein, the concentration of antibody in the intravenous infusion is between 10 mg/ml and 30 mg/ml.

In some embodiments of all the methods described herein, the subject is a pregnant woman. In some embodiments of all the methods described herein, the dose is based on the weight of the pregnant woman at first dosing and is not adjusted upward based on weight gain by the pregnant woman. In some embodiments of all the methods described herein, the dose is dose per administration and is based on the weight of the pregnant woman at first dosing and is adjusted upward based on weight gain by the pregnant woman.

In some embodiments of all the methods described herein, the composition is administered at least every other week. In some embodiments of all the methods described herein, the composition is administered every other week. In some embodiments of all the methods described herein, the composition is administered at least every week. In some embodiments of all the methods described herein, the composition is administered every week.

In some embodiments of all the methods described herein, the subject is a pregnant woman and the first infusion is administered during the first trimester of pregnancy. In some embodiments of all the methods described herein, the subject is a pregnant woman and the first infusion is administered during the second trimester of pregnancy. In some embodiments of all the methods described herein, the subject is a pregnant woman and the first infusion is administered during the third trimester of pregnancy. In some embodiments of all the methods described herein, the subject is a pregnant woman and the pregnant woman has an obstetrical history of severe fetal anemia. In some embodiments of all the methods described herein, the subject is a pregnant woman and the pregnant woman has an obstetrical history of hemolytic disease of the fetus and newborn. In some embodiments of all the methods described herein, the subject is a pregnant woman and the pregnant woman has an elevated anti-RhD, anti-Rhc or anti-Kell immunoglobulin alloantibody titer. In some embodiments of all the methods described herein, the subject is a pregnant woman and the pregnant woman has an elevated anti-Rhc or anti-Kell immunoglobulin alloantibody titer. In some embodiments of all the methods described herein, the subject is a pregnant woman and the pregnant woman has an elevated immunoglobulin alloantibody titer for one or more antibodies selected from the group consisting of anti-Lu^{a}, Lu^{b}, Bg, Kn^{a}, Yt^{a}, E. C. K. C^{w}, Fy^{a}, cE, ce, D, Ce, cE, K, Kp^{a}, Kp^{b}, Fy^{a}, M, N, S, Le^{a}, Le^{b}, Fy, Jk^{a}. Diego, P and Mi^{a}/Mur. In some embodiments of all the methods described herein, the subject is a pregnant woman and the pregnant woman has an obstetrical history of severe fetal anemia or stillbirth at ≤24 weeks gestation and elevated anti-D or anti-Kell IgG alloantibody titers and is pregnant with an antigen-positive fetus. In some embodiments of all the methods described herein, the subject is a pregnant woman and the first infusion is weeks 12 to 16 of pregnancy. In some embodiments of all the methods described herein, the subject is a pregnant woman and the first infusion is during week 14 of pregnancy.

In some embodiments of all the methods described herein, the infusion times are identical and takes place over 90 minutes or less, 60 minutes or less, 45 minutes or less, 30 minutes or less, 15 minutes or less, or 7 minutes or less. In some embodiments of all the methods described herein, the first infusion takes place over 90 minutes or less, 60 minutes or less, 45 minutes or less, 30 minutes or less, 15 minutes or less, and subsequent infusion times are reduced. In some embodiments of all the methods described herein, the first infusion takes place over 60 minutes and subsequent infusions take place over 45 minutes or less, 30 minutes or less, or 15 minutes or less; or the first infusion takes place over 45 minutes and subsequent infusions takes place over 30 minutes or less or 15 minutes or less; or the first infusion takes place over 30 minutes and subsequent infusions takes place over 15 minutes or less.

In some embodiments of all the methods described herein, the second fusion and the third fusion times are identical, takes place over 60 minutes or less, 45 minutes or less, 30 minutes or less, 15 minutes or less, or 7 minutes or less, and subsequent infusion times are reduced.

In some embodiments of all the methods described herein, the first infusion and the second fusion times are identical, take place over 90 minutes or less, 60 minutes or less, 45 minutes or less, 30 minutes or less, 15 minutes or less, and subsequent infusion times are reduced. In some embodiments of all the methods described herein, the first infusion and the second fusion both take place over 60 minutes and subsequent infusions take place over 45 minutes or less, 30 minutes or less, or 15 minutes or less; or the first infusion and the second fusion both take place over 45 minutes and subsequent infusions take place over 30 minutes or less or 15 minutes or less; or the first infusion and the second fusion both take place over 30 minutes and subsequent infusions take place over 15 minutes or less.

### Formulation

The composition for intravenous infusion is an aqueous composition that is physiologically compatible (e.g., buffered to a physiological pH and substantially isotonic. The composition can include, for example: sodium chloride, Trehalose, and surfactant polysorbate (PS) 80, and buffered agents. The composition can include both an ionic osmolyte stabilizer (sodium chloride) and non-ionic osmolyte stabilizer (trehalose).
Suitable formulation include (1) 25 mM sodium phosphate, 25 mM sodium chloride, 90.5 mg ml⁻¹ Trehalose, 0.01% polysorbate (PS) 80, and antibody at 10 or 30 mg ml⁻¹ buffered at pH 6.5; and (2) 25 mM sodium succinate, 25 mM sodium chloride, 90.5 mg ml⁻¹ Trehalose, 0.01% polysorbate (PS) 80, and antibody at 10 or 30 mg ml⁻¹ buffered at pH 6.6 or pH 6.5.

### Definitions

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit FcRn antigen-binding activity.

“Antibody fragments” comprise a portion of an intact antibody, preferably the antigen binding or variable region of the intact antibody. Examples of antibody fragments include Fab, Fab′, F(ab′)₂, and Fv fragments, diabodies, linear antibodies, single-chain antibody molecules, and multispecific antibodies.

As used herein, the term “isolated antibody” refers to an antibody which has been separated and/or recovered from a component of its manufacturing host cell environment. Contaminant components of its manufacturing host cell environment are materials which would interfere with research, diagnostic, or therapeutic uses of the antibody. Contaminant components may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or non-reducing conditions using, for example, Coomassie blue or silver stain. An isolated antibody includes the antibody in situ within recombinant cells. Ordinarily, however, an isolated antibody will be prepared by at least one purification step. A pharmaceutical preparation of an isolated antibody typically has less than 250 ppm (e.g., less than 200 ppm, 150 ppm. 100 ppm) of host cell proteins (HCP) as determined by an ELISA based HCP assay performed as recommended by an FDA “Guidance for Industry” document.

As used herein, the term “monoclonal antibody” refers to an antibody obtained from a population of substantially homogeneous antibodies, i.e., individual antibodies in the population have the same primary sequence except for possible naturally occurring mutations that may be present in minor amounts. Monoclonal antibodies are highly specific and directed against a single antigenic site (i.e., an epitope on human FcRn). In contrast to polyclonal antibody preparations which typically include different antibodies directed against different epitopes, each monoclonal antibody is directed against a single epitope on the antigen. The modifier “monoclonal” indicates the character of the antibody as being obtained from a substantially homogenous population of antibodies, and is not to be construed as requiring production of the antibody by any particular method.

As used herein, the terms “variable region” and “variable domain” refer to the portions of the light and heavy chains of an antibody that include amino acid sequences of complementary determining regions (CDRs, e.g., CDR L1, CDR L2, CDR L3, CDR H1, CDR H2, and CDR H3) and framework regions (FRs). According to the methods used in this disclosure, the amino acid positions assigned to CDRs and FRs are defined according to Kabat (Sequences of Proteins of Immunological Interest, 5th Ed. Public Health Service, National Institutes of Health, Bethesda, Md. (1991)). Using this numbering system, the actual linear amino acid sequence may contain fewer or additional amino acids corresponding to a shortening of, or insertion into, a CDR (defined further herein) or FR (defined further herein) of the variable region. For example, a heavy chain variable region may include a single inserted residue (i.e., residue 52a according to Kabat) after residue 52 of CDR H2 and inserted residues (i.e., residues 82a, 82b, 82c, etc. according to Kabat) after residue 82 of heavy chain FR. The Kabat numbering of residues may be determined for a given antibody by alignment at regions of homology of the sequence of the antibody with a “standard” Kabat numbered sequence.

As used herein, the terms “complementary determining regions” and “CDRs” refer to the regions of an antibody variable domain or variable region which are hypervariable in sequence and/or form structurally defined loops. A CDR is also known as a hypervariable region. The light chain and heavy chain variable regions each has three CDRs. The light chain variable region contains CDR L1, CDR L2, and CDR L3. The heavy chain variable region contains CDR H1, CDR H2, and CDR H3. Each CDR may include amino acid residues from a complementarity determining region as defined by Kabat (i.e. about residues 24-34 (CDR L1), 50-56 (CDR L2) and 89-97 (CDR L3) in the light chain variable region and about residues 31-35 (CDR H1), 50-65 (CDR H2) and 95-102 (CDR H3) in the heavy chain variable region.

As used herein, the term “FcRn” refers a neonatal Fc receptor that binds to the Fc region of an IgG antibody, e.g., an IgG1 antibody. An exemplary FcRn is human FcRn having UniProt ID No. P55899. Human FcRn is believed to be responsible for maintaining the half-life of IgG by binding and trafficking constitutively internalized IgG back to the cell surface for the recycling of IgG.

As used herein, the terms “affinity” and “binding affinity” refer to the strength of the binding interaction between two molecules. Generally, binding affinity refers to the strength of the sum total of non-covalent interactions between a single binding site of a molecule and its binding partner, such as an isolated antibody and its target (e.g., an isolated anti-FcRn antibody and a human FcRn). Unless indicated otherwise, binding affinity refers to intrinsic binding affinity, which reflects a 1:1 interaction between members of a binding pair. The binding affinity between two molecules is commonly described by the dissociation constant (K_{D}) or the affinity constant (K_{A}). Two molecules that have low binding affinity for each other generally bind slowly, tend to dissociate easily, and exhibit a large K_{D}. Two molecules that have high affinity for each other generally bind readily, tend to remain bound longer, and exhibit a small K_{D}. One method for determining the K_{D} of an antibody to human FcRn is described in Example 2 (“the SPR method”). Using this method the K_{D} of N022, N023, N024, N026, and N027 was 31, 31.4, 35.5, 36.5, and 19.3 pM, respectively.

As used herein, the term “inhibit IgG binding to FcRn” refers to the ability of an anti-FcRn antibody to block or inhibit the binding of IgG (e.g., IgG1) to human FcRn. In some embodiments, an anti-FcRn antibody binds FcRn, for example, at the site on human FcRn to which IgG binds. Thus, the anti-FcRn antibody is able to inhibit the binding of IgG (e.g., a subject's autoantibodies) to FcRn. In some embodiments, the molecule (e.g., an anti-FcRn antibody of the disclosure) substantially or completely inhibits binding to IgG. In some embodiments, the binding of IgG is reduced by 10%, 20%, 30%, 50%, 70%, 80%, 90%, 95%, or even 100%.

As used herein, the term “inhibit pathogenic antibody binding to FcRn” refers to the ability of an anti-FcRn antibody to block or inhibit the binding of a pathogenic antibody (e.g., pathogenic IgG antibody) to human FcRn. In some embodiments, an anti-FcRn antibody binds FcRn, for example, at the site on human FcRn to which the pathogenic antibody binds. Thus, the anti-FcRn antibody is able to inhibit the binding of pathogenic antibodies (e.g., pathogenic IgG antibodies) to FcRn. In some embodiments, the molecule (e.g., an anti-FcRn antibody) substantially or completely inhibits binding to pathogenic antibodies. In some embodiments, the binding of pathogenic antibodies to FcRn is reduced by 10%, 20%, 30%, 50%, 70%, 80%, 90%, 95%, or even 100%.

As used herein, the term “hydrophobic amino acid” refers to an amino acid having relatively low-water solubility. Hydrophobic amino acids include, but are not limited to, leucine, isoleucine, alanine, phenylalanine, valine, and proline. Particularly preferred hydrophobic amino acids in the present disclosure are alanine, leucine, isoleucine, and valine.

As used herein, the term “polar amino acid” refers to an amino acid having a chemical polarity in its side chain induced by atoms with different electronegativity. The polarity of a polar amino acid is dependent on the electronegativity between atoms in the side chain of the amino acid and the asymmetry of the structure of the side chain. Polar amino acids include, but are not limited to, serine, threonine, cysteine, methionine, tyrosine, tryptophan, asparagine, and glutamine. Particularly preferred polar amino acids in the present disclosure are serine, threonine, asparagine, glutamine, cysteine, and tyrosine.

As used herein, the term “acidic amino acid” refers to an amino acid whose side chain contains a carboxylic acid group having a pKa between 3.5 and 4.5. In some embodiments, acidic amino acids are aspartic acid and glutamic acid.

As used herein, the term “basic amino acid” refers to an amino acid whose side chain contains an amino group having a pKa between 9.5 and 13. In some embodiments, basic amino acids are histidine, lysine, and arginine.

As used herein, the term “percent (%) identity” refers to the percentage of amino acid (or nucleic acid) residues of a candidate sequence, e.g., an anti-FcRn antibody of the disclosure, that are identical to the amino acid (or nucleic acid) residues of a reference sequence, e.g., a wild-type anti-FcRn antibody, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular, embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position. A position may be altered by a substitution, deletion, or insertion. A substitution, deletion, or insertion may comprise a certain number of amino acids, (e.g., 1, 2, 3, 4, 5, 6, 7, 8, 9, 10, 11, 12, 13, 14, 15, or more). When describing a substitution, deletion, or insertion of no more than n amino acids, this is meant that the substitution, deletion, or insertion comprises, e.g., 1, 2, 3, 4, 5, 6, 7, 8, 9, 10, 11, 12, 13, 14, 15, or n amino acids. The number or substitutions, deletions, or insertions can comprise a percent of the total sequence (e.g., 1%, 5%, 10%, 15%, 20%, or more) where the number of substitutions, deletions, or insertions alters 5%, 10%, 15%, 20% or more, of the amino acids in the total sequence.

As used herein, the term “fetal and neonatal alloimmune and/or autoimmune disorder” refers to an immune disorder in a fetus and/or neonate that is caused by the transplacental transfer of maternal antibodies (e.g., pathogenic maternal antibodies) directed against fetal and/or neonate antigens. For example, a pregnant subject's antibodies (e.g., pathogenic antibodies) may react against antigens in the fetus (e.g., antigens the fetus inherited from the fetus' father). Examples of fetal and neonatal alloimmune and/or autoimmune disorders are provided herein.

As used herein, the term “pathogenic antibody” refers to an antibody that causes one or more immune diseases or disorders in a subject (e.g., a pregnant subject), a fetus in a pregnant subject, and/or a neonate. In some embodiments, pathogenic antibodies are autoantibodies produced in a subject (e.g., a pregnant subject) against one or more of the subject's own proteins, thus causing autoimmune diseases or disorders in the subject. In some embodiments, pathogenic antibodies in a pregnant subject may transfer through the placenta to the fetus and react against antigens from the fetus (e.g., antigens that the fetus inherited from the fetus' father), thus causing, e.g., fetal and neonatal alloimmune and/or autoimmune disorders.

As used herein, the term “antibody-mediated enhancement of viral disease” refers to a viral disease in which antibodies can facilitate viral entry into host cells, thus leading to increased or enhanced infectivity in the cells. In some embodiments, an antibody may bind to a viral surface protein and the antibody/virus complex may bind to an FcRn receptor on a cell surface through interaction between the antibody and the receptor. Subsequently, the antibody/virus complex may be internalized into the cell.

As used herein, the term “gestational age” describes how far along the pregnancy is. The gestational age can be described in terms of weeks. Methods of determining gestational age are known in the art (e.g., Committee on Obstetric Practice American Institute of Ultrasound in Medicine Society for Maternal-Fetal Medicine, Committee Opinion. Number 700. May 2017; which is incorporated herein in its entirety). In some instances, the gestational age can be determined by ultrasound, weeks since first day of last menstrual period (LMP), or combinations thereof.

As used herein, the term “pharmaceutical composition” refers to a medicinal or pharmaceutical formulation that contains an active ingredient as well as one or more excipients and diluents to enable the active ingredient suitable for the method of administration. The pharmaceutical composition of the present disclosure includes pharmaceutically acceptable components that are compatible with the anti-FcRn antibody. The pharmaceutical composition may be in aqueous form for intravenous or subcutaneous administration or in tablet or capsule form for oral administration.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present disclosure, the pharmaceutically acceptable carrier must provide adequate pharmaceutical stability to the Fc construct. The nature of the carrier differs with the mode of administration. For example, for intravenous administration, an aqueous solution carrier is generally used; for oral administration, a solid carrier is preferred.

As used herein, the term “therapeutically effective amount” refers to an amount, e.g., pharmaceutical dose, effective in inducing a desired biological effect in a subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents.

As used herein, the term “no more than” refers to an amount that is less than equal to. This may be an amount in integers. For example, no more than two substitutions can refer to 0, 1, or 2 substitutions.

As used herein, the terms “treatment” or “treating” refer to reducing, decreasing, decreasing the risk of, or decreasing the side effects of a particular disease or condition. Reducing, decreasing, decreasing the risk of, or decreasing the side effects of are relative to a subject who did not receive treatment, e,g, a control, a baseline, or a known control level or measurement.

### DESCRIPTION OF THE DRAWINGS

FIG. 1 includes two graphs and a table that show IgG competitive binding of antibodies N022-N024, N026, and N027 to human or cynomolgus monkey FcRn at pH 6.0.

FIG. 2 includes graphs that show the effects of antibodies N023, N024, N026, and N027 on IgG catabolism in mice.

FIG. 3 includes graphs that show the dose-dependent effects of antibody N027 on IgG levels and target occupancy in mice.

FIGS. 4A-4C includes graphs that show the selective induction of IgG catabolism and target occupancy in cynomolgus monkeys following administration of different doses of antibody N027.

FIG. 5 includes an experimental timeline and two graphs that show the efficacy of N027 in a mouse chronic idiopathic thrombocytopenia purpura (ITP) model.

### DETAILED DESCRIPTION

Described herein is a method for intravenous (IV) administration of anti-FcRn antibodies. The IV antibodies can be relatively rapidly, yet safely.

### I. Anti-FcRn Antibodies

In general, the disclosure features intravenous administration of certain isolated antibodies that bind to the human FcRn with high affinity. An anti-FcRn antibody refers to an antibody that can bind to human FcRn and inhibit IgG (e.g., IgG autoantibodies) binding to FcRn.

In one aspect, the disclosure features intravenous administration of an isolated antibody capable of binding to human FcRn. In some embodiments, the isolated antibody contains: (1) a light chain variable region that includes a CDR L1, a CDR L2, and a CDR L3 and (2) a heavy chain variable region that includes a CDR H1, a CDR H2, and a CDR H3, wherein the CDR L1 comprises TGTGSDVGSYNLVS (SEQ ID NO: 1), the CDR L2 comprises a GDSERPS (SEQ ID NO: 2), the CDR L3 comprises of SSYAGSGIYV (SEQ ID NO: 3), the CDR H1 comprises TYAMG (SEQ ID NO: 4), DYAMG (SEQ ID NO: 5), or NYAMG (SEQ ID NO: 6), the CDR H2 comprises SIGSSGAQTRYADS (SEQ ID NO: 7), SIGASGSQTRYADS (SEQ ID NO: 8), SIGASGAQTRYADS (SEQ ID NO: 9), or SIGASGGQTRYADS (SEQ ID NO: 10), and the CDR H3 comprises LAIGDSY (SEQ ID NO: 11). In some embodiments, the antibody comprises a heavy chain comprising a sequence having at least 95%, 97%, 99%, or 100% identity to the sequence of any one of SEQ ID NOs: 20-24 and a light chain comprises a sequence having at least 95%, 97%, 99%, or 100% identity to the sequence of SEQ ID NO: 19. In some embodiments, the antibody heavy chain comprises the amino acid sequence of any of SEQ ID Nos: 20-24 with amino acid other than N at position 296 of SEQ ID NOs: 20-24. In some embodiments, the antibody heavy chain comprises the amino acid sequence of SEQ ID NO:24 with one or more of the following amino acid substitutions: A23V, S30R, L80V, A84T, E85D, A93V, relative to the sequence of SEQ ID NO: 24. In some embodiments, the antibody light chain comprises the amino acid sequence of SEQ ID NO: 19 with one or more of the following amino acid substitutions: Q38H, V58I, and G99D, relative to the sequence of SEQ ID NO: 19. In some embodiments, the antibody heavy chain does not contain a C-terminal lysine. In some embodiments, the heavy chain comprises SEQ ID NO:24 or a variant of SEQ ID NO:24 wherein the amino acid at 296 is other than N. In some embodiments, the antibody binds human FcRn with a K_{D} of less than 200, 150, 100, 50, or 40 pM. In some embodiments, the antibody binds human FcRn with a K_{D} that is less than or equal to that of an antibody having the light chain variable region and heavy chain variable region of N022, N023, N024, N026, or N027, and further having the same Fc region as the antibody being compared. In some embodiments, the antibody is an IgG1 isotype. In some embodiments, the antibody is fully human. In some embodiments, the antibody is aglycosylated at position N297 according to EU numbering. In some cases less the antibody composition administered is less than 20%, 10% or 5% wt/wt antibody that is glyclosylated on the Fc domain.

Table 1 shows the amino acid sequences of the light and heavy chain complementary determining regions (CDRs) of some exemplary anti-FcRn antibodies of the disclosure.

TABLE 1

| Anti- | | | | | | |
|---|---|---|---|---|---|---|
| FcRn | | | | | | |
| anti- | | | | | | |
| body | CDR L1 | CDR L2 | CDR L3 | CDR H1 | CDR H2 | CDR H3 |
| N022 | TGTG | GDSE | SSYA | TYAM | SIGS | LAIG |
| | SDVG | RPS | GSGI | G | SGAQ | DSY |
| | SYNL | (SEQ | YV | (SEQ | TRYA | (SEQ |
| | VS | ID | (SEQ | ID | DS | ID |
| | (SEQ | NO: 2) | ID | NO: 4) | (SEQ | NO: 11) |
| | ID | | NO: 3) | | ID | |
| | NO: 1) | | | | NO: 7) | |
| N023 | TGTG | GDSE | SSYA | DYAM | SIGA | LAIG |
| | SDVG | RPS | GSGI | G | SGSQ | DSY |
| | SYNL | (SEQ | YV | (SEQ | TRYA | (SEQ |
| | VS | ID | (SEQ | ID | DS | ID |
| | (SEQ | NO: 2) | ID | NO: 5) | (SEQ | NO: 11) |
| | ID | | NO: 3) | | ID | |
| | NO: 1) | | | | NO: 8) | |
| N024 | TGTG | GDSE | SSYA | NYAM | SIGA | LAIG |
| | SDVG | RPS | GSGI | G | SGAQ | DSY |
| | SYNL | (SEQ | YV | (SEQ | TRYA | (SEQ |
| | VS | ID | (SEQ | ID | DS | ID |
| | (SEQ | NO: 2) | ID | NO: 6) | (SEQ | NO: 11) |
| | ID | | NO: 3) | | ID | |
| | NO: 1) | | | | NO: 9) | |
| N026 | TGTG | GDSE | SSYA | TYAM | SIGA | LAIG |
| | SDVG | RPS | GSGI | G | SGGQ | DSY |
| | SYNL | (SEQ | YV | (SEQ | TRYA | (SEQ |
| | VS | ID | (SEQ | ID | DS | ID |
| | (SEQ | NO: 2) | ID | NO: 4) | (SEQ | NO: 11) |
| | ID | | NO: 3) | | ID | |
| | NO: 1) | | | | NO: 10) | |
| N027 | TGTG | GDSE | SSYA | TYAM | SIGA | LAIG |
| | SDVG | RPS | GSGI | G | SGSQ | DSY |
| | SYNL | (SEQ | YV | (SEQ | TRYA | (SEQ |
| | VS | ID | (SEQ | ID | DS | ID |
| | (SEQ | NO: 2) | ID | NO: 4) | (SEQ | NO: 11) |
| | ID | | NO: 3) | | ID | |
| | NO: 1) | | | | NO: 8) | |

Table 2 shows the SEQ ID NOs of the light and heavy chains of these exemplary anti-FcRn antibodies of the disclosure.

TABLE 2

| Anti-FcRn | | |
|---|---|---|
| antibody | Light Chain | Heavy Chain |
| N022 | SEQ ID NO: 19 | SEQ ID NO: 20 |
| N023 | | SEQ ID NO: 21 |
| N024 | | SEQ ID NO: 22 |
| N026 | | SEQ ID NO: 23 |
| N027 | | SEQ ID NO: 24 |

Furthermore, in any of the anti-FcRn antibodies described herein, the heavy chain of the antibody comprises a sequence having at least 95%, 97%, 99%, or 100% identity to the sequence of any one of SEQ ID NOs: 20-24. In any of the anti-FcRn antibodies described herein, the light chain comprises a sequence having at least 95%, 97%, 99%, or 100% identity to the sequence of SEQ ID NO: 19. In any of the anti-FcRn antibodies described herein, the heavy chain variable region of the antibody comprises any one of SEQ ID NOs: 20-24. In any of the anti-FcRn antibodies described herein, the light chain variable region of the antibody comprises any one of SEQ I D NOs: 19. In some embodiments, the antibody comprises a heavy chain comprising a sequence having at least 95%, 97%, 99%, or 100% identity to the sequence of any one of SEQ ID NOs: 20-24 and a light chain comprises a sequence having at least 95%, 97%, 99%, or 100% identity to the sequence of SEQ ID NO: 19. In some embodiments, the antibody heavy chain comprises the amino acid sequence of any of SEQ ID Nos: 20-24 with amino acid other than N at position 296 of SEQ ID NOs: 20-24. In some embodiments, the antibody heavy chain comprises the amino acid sequence of SEQ ID NO:24 with one or more of the following amino acid substitutions: A23V, S30R, L80V, A84T, E85D, A93V, relative to the sequence of SEQ ID NO: 24. In some embodiments, the antibody light chain comprises the amino acid sequence of SEQ ID NO: 19 with one or more of the following amino acid substitutions: Q38H, V58I, and G99D, relative to the sequence of SEQ ID NO: 19. In some embodiments, the antibody heavy chain does not contain a C-terminal lysine. In some embodiments, the heavy chain comprises SEQ ID NO:24 or a variant of SEQ ID NO:24 wherein the amino acid at 296 is other than N.

In some embodiments, the light chain variable region comprises a CDR L1, a CDR L2, and a CDR L3 and a heavy chain variable region that includes a CDR H1, a CDR H2, and a CDR H3, wherein the CDR L1 comprises a sequence having no more than two amino acid substitutions relative to the sequence of TGTGSDVGSYNLVS (SEQ ID NO: 1), the CDR L2 comprises a sequence having no more than one amino acid substitutions relative to the sequence of GDSERPS (SEQ ID NO: 2), the CDR L3 comprises a sequence having no more than one amino acid substitutions relative to the sequence of SSYAGSGIYV (SEQ ID NO: 3), the CDR H1 comprises a sequence having no more than one amino acid substitutions relative to the sequence of TYAMG (SEQ ID NO: 4), DYAMG (SEQ ID NO: 5), or NYAMG (SEQ ID NO: 6), the CDR H2 comprises a sequence having no more than two amino acid substitutions relative to the sequence of SIGSSGAQTRYADS (SEQ ID NO: 7), SIGASGSQTRYADS (SEQ ID NO: 8), SIGASGAQTRYADS (SEQ ID NO: 9), or SIGASGGQTRYADS (SEQ ID NO: 10), and the CDR H3 comprises a sequence having no more than one amino acid substitutions relative to the sequence of LAIGDSY (SEQ ID NO: 11).

The antibodies may further contain amino acid substitutions, additions, and/or deletions outside of the CDRs (i.e., in framework regions (FRs)). In some embodiments, the antibodies may further include any one or more of the following amino acid substitutions: A23V, S30R, L80V, A84T, E85D, A93V, relative to the sequence of any one of SEQ ID NOs: 20-24, and Q38H, V58I, and G99D, relative to the sequence of SEQ ID NO: 19 (Numbering according the EU system)

The antibodies may further contain amino acid substitutions, additions, and/or deletions outside of the CDRs (i.e., in framework regions (FRs)). An amino acid substitution, addition, and/or deletion can be a substitution, addition, and/or deletion of one or more amino acids (e.g., 1, 2, 3, 4, 5, 6, 7, 8, or more). An amino acid substitution, addition, and/or deletion can be a substitution, addition, and/or deletion of eight or fewer, seven or fewer, six or fewer, five or fewer, four or fewer, three or fewer, or two or fewer single amino acids. In some embodiments, the antibodies may further include any one or more of the following amino acid substitutions: A23V, S30R, L80V, A84T, E85D, A93V, relative to the sequence of any one of SEQ ID NOs: 20-24, and Q38H, V58I, and G99D, relative to the sequence of SEQ ID NO: 19 (Numbering according to the EU system).

In some embodiments, the antibodies may include amino acid substitutions, additions, and/or deletions in the constant regions (e.g., Fc region) of the antibody that, e.g., lead to decreased effector function, e.g., decreased complement-dependent cytolysis (CDC), antibody-dependent cell-mediated cytolysis (ADCC), and/or antibody-dependent cell-mediated phagocytosis (ADCP), and/or decreased B-cell killing. The constant regions are not involved directly in binding an antibody to its target, but exhibit various effector functions, such as participation of the antibody in antibody-dependent cellular toxicity. In some embodiments, the antibodies are characterized by decreased binding (i.e., absence of binding) to human complement factor C1q and/or human Fc receptor on natural killer (NK) cells. In other embodiments, the antibodies are characterized by decreased binding (i.e., absence of binding) to human FcγRI, FcγRIIA, and/or FcγRIIIA. To alter or reduce an antibody-dependent effector function, such as CDC, ADCC, ADCP, and/or B-cell killing, antibodies may be of the IgG class and contain one or more amino acid substitutions E233, L234, G236, D265, D270, N297, E318, K320, K322, A327, A330, P331, and/or P329 (numbering according to the EU System). In some embodiments, the antibodies contain the mutations L234A/L235A or D265A/N297A. In some cases, an anti-FcRn antibody is aglycosylated at position 297. In some cases, an anti-FcRn antibody does not have an N at position 297 (EU numbering) in any one of SEQ ID NOs: 20-24, such that the antibody is aglycosylated at that position. In some cases, the anti-FcRn antibody has a modified sequence such that the N at position 297 (EU numbering) is not glycosylated. The resulting effectorless antibody shows very little binding to complement or Fc receptors (i.e., complement C1q binding), indicating low CDC potential.

In other embodiments, the antibodies may include those having specific amino acid changes that improve stability of the antibody.

Moreover, in other embodiments, to minimize potential immunogenicity, some antibodies of the disclosure, e.g., N024, N026, and N027, may undergo an allotype change from G1 m17.1 to G1 m17 by substituting amino acids D355 and L357 (relative to the sequence of any one of SEQ ID NOs: 20-24) to glutamic acid and methionine, respectively.

In other embodiments, the antibodies of the disclosure, e.g., N022-N024, N026, and N027, do not contain a C-terminal lysine at residue 446, relative to the sequence of any one of SEQ ID NOs: 20-24.

Without being bound by theory, it is believed that the anti-FcRn antibodies compete with and inhibit the binding of IgG to human FcRn. Epitope mapping by hydrogen-deuterium exchange of the antibodies indicates that the antibodies bind to an epitope on FcRn located in and/or adjacent to the Fc-FcRn interaction interface, which suggests that the antibodies block IgG binding to FcRn by direction inhibition. Furthermore, the epitope-mapped binding site is distant from the albumin-binding site of FcRn. Accordingly, serum albumin-binding should not be inhibited and serum albumin levels should not be decreased. Indeed, experimental evidence shows mouse albumin levels remained constant after anti-FcRn antibody administration, indicating that albumin recycling is not disturbed by antibody binding to FcRn.

### II. FcRn Inhibition

FcRn is a type I transmembrane protein that functions as an IgG- and serum albumin-binding, intracellular vesicular trafficking protein. FcRn is expressed in endothelial cells, luminal epithelial cells, hepatocytes, podocytes, granulocytes, monocytes, macrophages, dendritic cells, and NK cells, but not on B or T cells. FcRn maintains the half-life of IgG by binding and trafficking constitutively internalized IgG back to the cell surface. Binding of both Fc and serum albumin by FcRn occurs in the early endosome at pH 6.0, followed by sorting of the FcRn into vesicles, which traffic the FcRn-bound IgG or albumin back to the cell surface where FcRn rapidly releases the IgG or albumin at pH 7.4. This trafficking cycle maintains the half-life of IgG and albumin by recycling both into the circulation and preventing trafficking to the lysosomes for degradation. FcRn also captures internalized IgG Fc in epithelial cells and transports them bidirectionally to the opposing apical or basolateral membranes. This function allows IgG to traffic to the lumen of organs such as the gastrointestinal tract or the transport of IgG or IgG-antigen complexes from the lumen to the vasculature or lymphoid tissues in the stromal layers.

In order to study the contribution of FcRn to IgG homeostasis, mice have been engineered so that parts of the light and heavy chains of FcRn have been “knocked out” so that these proteins are not expressed (Junghans et al., Proc Natl Acad Sci USA 93:5512, 1996). In these mice, the serum half-life and concentrations of IgG were dramatically reduced, suggesting an FcRn-dependent mechanism of IgG homeostasis. Studies in rodent models, such as the one discussed above, suggest that blockage of FcRn can increase IgG catabolism, including that of pathogenic autoantibodies, thereby inhibiting disease (e.g., an autoimmune disease) development. FcRn may also contribute to antigen presentation through trafficking of immune complexes to antigen degradation and MHC loading compartments.

The present disclosure provides isolated anti-FcRn antibodies that bind to human FcRn with high affinity. The anti-FcRn antibodies compete with and effectively inhibit the binding of other anti-FcRn antibodies (e.g., IgG, IgG autoantibodies) to FcRn, thereby increasing the catabolism and decreasing the half-life of other anti-FcRn antibodies (e.g., IgG, IgG autoantibodies). The anti-FcRn antibodies may be used in a method of treating or reducing immune complex-based activation of an immune response in a subject, such as an immune response caused by autoantibodies in an autoimmune disease.

Placental transfer of maternal IgG antibodies to the fetus is an important FcRn-dependent mechanism that provides protection to the neonate while his/her humoral response is inefficient. During fetal life, FcRn in the syncytiotrophoblast layers of the placenta is responsible for the transfer of maternal IgG antibodies to the fetus. Pathogenic maternal antibodies (e.g., pathogenic maternal IgG antibodies) may also cross the placenta by binding to FcRn and cause alloimmune disorders and/or autoimmune disorders in the fetus and neonate. In some embodiments, pathogenic antibodies in the pregnant subject cause a fetal and neonatal alloimmune and/or autoimmune disorder in a fetus in the pregnant subject. The anti-FcRn antibodies described herein (e.g., N022-N024, N026, and N027, preferably N027 and/or N024) may compete with and inhibit the binding of maternal pathogenic antibodies (e.g., maternal pathogenic IgG antibodies) to FcRn, thereby increasing the catabolism and decreasing the half-life of these pathogenic antibodies.

The present disclosure provides isolated anti-FcRn antibodies that bind to human FcRn. The anti-FcRn antibodies may compete with and inhibit the binding of other anti-FcRn antibodies (e.g., IgG, IgG autoantibodies) to FcRn, thereby increasing the catabolism and decreasing the half-life of other anti-FcRn antibodies (e.g., IgG, IgG autoantibodies). The anti-FcRn antibodies may be used in a method of treating or reducing immune complex-based activation of an immune response in a subject, such as an immune response caused by autoantibodies in an autoimmune disease. Reducing an immune response may be described as reducing an immune response relative to a subject who does not receive treatment (e.g., a control subject). The anti-FcRn antibodies may also be used in methods of decreasing pathogenic antibody transport (e.g., pathogenic maternal IgG antibody transport) across the placenta of a pregnant subject, increasing pathogenic antibody catabolism in a pregnant subject, and treating an antibody-mediated enhancement of viral disease in a fetus or a neonate by administering to a pregnant subject an isolated antibody that binds to human FcRn. Decreasing pathogenic antibody transport across the placenta of a pregnant subject, may be described as decreasing pathogenic antibody transport relative to a subject who does not receive treatment (e.g., a control subject).

### III. Vectors, Host Cells, and Antibody Production

The anti-FcRn antibodies can be produced from a host cell. A host cell refers to a vehicle that includes the necessary cellular components, e.g., organelles, needed to express the polypeptides and constructs described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, etc). The choice of nucleic acid vectors depends in part on the host cells to be used. Generally, preferred host cells are of either prokaryotic (e.g., bacterial) or eukaryotic (e.g., mammalian) origin.

Nucleic Acid Vector Construction and Host Cells

A nucleic acid sequence encoding the amino acid sequence of an anti-FcRn antibody may be prepared by a variety of methods known in the art. These methods include, but are not limited to, oligonucleotide-mediated (or site-directed) mutagenesis and PCR mutagenesis. A nucleic acid molecule encoding an anti-FcRn antibody may be obtained using standard techniques, e.g., gene synthesis. Alternatively, a nucleic acid molecule encoding a wild-type anti-FcRn antibody may be mutated to contain specific amino acid substitutions using standard techniques in the art, e.g., QuikChange™ mutagenesis. Nucleic acid molecules can be synthesized using a nucleotide synthesizer or PCR techniques.

Nucleic acid sequences encoding anti-FcRn antibodies may be inserted into a vector capable of replicating and expressing the nucleic acid molecules in prokaryotic or eukaryotic host cells. Many vectors are available in the art and can be used for the purpose of the disclosure. Each vector may contain various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells are used as host cells for the disclosure. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7O3O, and HsS78Bst cells. In other embodiments, E. coli cells are used as host cells for the disclosure. Examples of E. coli strains include, but are not limited to, E. coli 294 (ATCC® 31,446), E. coli λ 1776 (ATCC® 31,537, E. coli BL21 (DE3) (ATCC® BAA-1025), and E. coli RV308 (ATCC® 31,608). Different host cells have characteristic and specific mechanisms for the posttranslational processing and modification of protein products. Appropriate cell lines or host systems may be chosen to ensure the correct modification and processing of the anti-FcRn antibody expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 (Jul. 20, 2004) and Vladimir Voynov and Justin A. Caravella (eds.) Therapeutic Proteins: Methods and Protocols (Methods in Molecular Biology) Humana Press; 2nd ed. 2012 (Jun. 28, 2012).

Protein Production, Recovery, and Purification

Host cells used to produce the anti-FcRn antibodies may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels, such as 5 to 10% (preferably 8%). The pH of the medium is generally from about 6.8 to 7.4, e.g., 7.0, depending mainly on the host organism. If an inducible promoter is used in the expression vector of the disclosure, protein expression is induced under conditions suitable for the activation of the promoter.

Protein recovery typically involves disrupting the host cell, generally by such means as osmotic shock, sonication, or lysis. Once the cells are disrupted, cell debris may be removed by centrifugation or filtration. The proteins may be further purified. An anti-FcRn antibody may be purified by any method known in the art of protein purification, for example, by protein A affinity, other chromatography (e.g., ion exchange, affinity, and size-exclusion column chromatography), centrifugation, differential solubility, or by any other standard technique for the purification of proteins. (see Process Scale Purification of Antibodies, Uwe Gottschalk (ed.) John Wiley & Sons, Inc., 2009). In some instances, an anti-FcRn antibody can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from the influenza hemagglutinin protein.

Alternatively, anti-FcRn antibodies can be produced by the cells of a subject (e.g., a human), e.g., in the context of therapy, by administrating a vector (e.g., a retroviral vector, adenoviral vector, poxviral vector (e.g., vaccinia viral vector, such as Modified Vaccinia Ankara (MVA)), adeno-associated viral vector, and alphaviral vector) containing a nucleic acid molecule encoding the anti-FcRn antibody of the disclosure. The vector, once inside a cell of the subject (e.g., by transformation, transfection, electroporation, calcium phosphate precipitation, direct microinjection, infection, etc) will promote expression of the anti-FcRn antibody, which is then secreted from the cell. If treatment of a disease or disorder is the desired outcome, no further action may be required. If collection of the protein is desired, blood may be collected from the subject and the protein purified from the blood by methods known in the art.

### IV. Pharmaceutical Compositions and Preparations

The disclosure features pharmaceutical compositions that include one or more anti-FcRn antibodies described herein. In some embodiments, pharmaceutical compositions contain one or more antibodies of the disclosure, e.g., N022-N024, N026, and N027, as the therapeutic proteins. In other embodiments, pharmaceutical compositions containing one or more antibodies of the disclosure, e.g., N022-N024, N026, and N027, may be used in combination with other agents (e.g., therapeutic biologics and/or small molecules) or compositions in a therapy. In addition to a therapeutically effective amount of the antibody, the pharmaceutical compositions may contain one or more pharmaceutically acceptable carriers or excipients, which can be formulated by methods known to those skilled in the art.

Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. Pharmaceutical compositions can be administered parenterally in the form of an injectable formulation. Pharmaceutical compositions for injection (i.e., intravenous injection) can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006).

The pharmaceutical composition may be formed in a unit dose form as needed. The amount of active component, e.g., one or more anti-FcRn antibodies (e.g., N022-N024, N026, and N027, preferably N027 and/or N024), included in the pharmaceutical preparations is such that a suitable dose within the designated range is provided (e.g., a dose within the range of 0.01-500 mg/kg of body weight).

In some embodiments, formulations can be prepared with different concentrations of sodium chloride, Trehalose, and surfactant polysorbate (PS) 80, buffered agents and buffered at different pH (pH 5 to 8). In some embodiments, the compositions include both an ionic osmolyte stabilizer (sodium chloride) and non-ionic osmolyte stabilizer (trehalose). The stability of the formulations and compositions can be assessed over time by appearance, pH, protein concentration, size purity, charge distribution, and thermal stability. These stability parameters can be measured by analytical techniques including pH, UV-Vis, size exclusion chromatography, ion exchange chromatography, CE-SDS, and differential scanning calorimetry.

In various embodiments, formulations can comprise: (1) 25 mM sodium phosphate, 25 mM sodium chloride, 90.5 mg ml⁻¹ Trehalose, 0.01% polysorbate (PS) 80, and an antibody disclosed herein at 10 or 30 mg ml⁻¹ buffered at pH 6.5; and (2) 25 mM sodium succinate, 25 mM sodium chloride, 90.5 mg ml⁻¹ Trehalose, 0.01% polysorbate (PS) 80, and an antibody disclosed herein at 10 or 30 mg ml⁻¹ buffered at pH 6.6. The stability of the aforementioned two formulations can be further tested in presence of select mechanical, thermal, and chemical stresses. In some embodiments, the stability of the composition can be maintained for more than 30 months for the formulation (1) 25 mM sodium phosphate, 25 mM sodium chloride, 90.5 mg ml-1 Trehalose, 0.01% polysorbate (PS) 80, and antibody at 10 or 30 mg ml-1 buffered at pH 6.5. In various embodiments, formulations can comprise 25 mM sodium phosphate, 25 mM sodium chloride, 90.5 mg ml-1 Trehalose, and an antibody disclosed herein, buffered at pH 6.5 with differing amounts of polysorbate 80. In some embodiments, a pharmaceutical composition comprises: an antibody disclosed herein with up to 5 single amino acid insertions, substitutions or deletions at 10 or 30 mg/ml, 20-30 mM sodium phosphate, 20-30 mM sodium chloride, 80-100 mg/ml Trehalose, and 0.10-0.005% w/v Polysorbate 80, buffered at pH 6.5.

### V. Routes, Dosage, and Administration

Pharmaceutical compositions that contain one or more anti-FcRn antibodies (e.g., N022-N024, N026, and N027, preferably N027 and/or N024) as the therapeutic proteins may be formulated for intravenous administration.

The dosage of the pharmaceutical compositions depends on factors including the route of administration, the disease to be treated, and physical characteristics, e.g., age, weight, general health, of the subject. Typically, the amount of an anti-FcRn antibody (e.g., any one of N022-N024, N026, and N027, preferably N027 or N024) contained within a single dose may be an amount that effectively prevents, delays, or treats the disease without inducing significant toxicity. A pharmaceutical composition may include a dosage of an anti-FcRn antibody ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 60, 100, 150, 200, 250, 300, 350, 400, 450, or 500 mg/kg) and, in a more specific embodiment, about 1 to about 100 mg/kg and, in a more specific embodiment, about 1 to about 50 mg/kg and, in another embodiment, about 30 to 60 mg/kg. The dosage may be adapted by the physician in accordance with conventional factors such as the extent of the disease and different parameters of the subject. Additionally, the dosage may be adapted by the physician in accordance with factors such as gestational age, preparation for birth, weight gain of woman, and/or length of pregnancy.

In some cases, the compositions and pharmaceutical compositions described herein are administered to a pregnant woman throughout pregnancy. In some cases, the compositions and pharmaceutical compositions described herein are administered to a pregnant woman for around 5-25 weeks during pregnancy (e.g., around 5, 6, 7, 8, 9, 10, 11, 12, 13, 14, 15, 16, 17, 18, 19, 20, 21, 22, 23, 24, or 25 weeks). In some instances, administration of the compositions and pharmaceutical compositions ceases after around gestational age 34 (week 34) (E.g., after week 34, 35, 36, or 37). In some instances, IVIG is administered to the pregnant woman after cessation of administration of the compositions and pharmaceutical compositions. In some instances, IVIG is administered between around 3-15 days (e.g., 3, 4, 5, 6, 7, 8, 9, 10, 11, 12, 13, 14, or 15 days) after cessation of administration of the compositions and pharmaceutical compositions. In some cases, the time of IVIG administration after cessation of administration of the compositions and pharmaceutical compositions is adapted in accordance with factors such as weight gain of woman. In some instances, the compositions and pharmaceutical compositions described herein are first administered after gestational age 12 (e.g. after 12, 13, 14, 15, 16, 17, 18, 19, 20, 21, 22, 23, 24, 25, 26, 27, 28, 29, or 30). In some cases they are administered during the pregnancy between gestational age 14 and 26 (e.g., 14 and 25; 15 and 25; or 15 and 26, etc.). In some cases they are administered during the pregnancy between gestational age 12 and 36 (e.g., 12 and 36; 12 and 35; 12 and 34; 13 and 36; 13 and 35; 13 and 34; 14 and 36; 14 and 35; 14 and 34; 15 and 36; 15 and 35; 15 and 34; 16 and 36; 16 and 35; or 16 and 34; etc.).

The pharmaceutical compositions are administered in a manner compatible with the dosage formulation and in such amount as is therapeutically effective to result in an improvement or remediation of the symptoms. Pharmaceutical compositions that contain an anti-FcRn antibody (e.g., any one of N022-N024, N026, and N027, preferably N027 or N024) may be administered to a subject in need thereof, for example, one or more times (e.g., 1-10 times or more) daily, weekly, every two weeks, every four weeks, monthly, twice a month, biannually, annually, or as medically necessary. Dosages may be provided in either a single or multiple dosage regimens. The timing between administrations may decrease as the medical condition improves or increase as the health of the patient declines.

The pharmaceutical compositions are administered in a manner and rate compatible with the dosage formulation. In some cases, the subject receives a single dose of 30 or 60 mg/kg antibody by intravenous infusion over 90 minutes or less. In some cases, the intravenous infusion takes please over 60 minutes or less, 45 minutes or less, 30 minutes or less, 15 minutes or less, or 7 minutes or less. In some

In some embodiments, the subject receives a dose of 30 mg/kg antibody by intravenous infusion over 15 minutes. In some embodiments, the subject receives a dose of 30 mg/kg antibody by intravenous infusion over 30 minutes. In some embodiments, the subject receives a dose of 45 mg/kg antibody by intravenous infusion over 15 minutes. In some embodiments, the subject receives a dose of 45 mg/kg antibody by intravenous infusion over 30 minutes. In some embodiments, the subject receives a dose of 60 mg/kg antibody by intravenous infusion over 30 minutes. In some embodiments, the subject receives a dose of 30 mg/kg antibody by intravenous infusion over 60 minutes. In some embodiments, the subject receives a dose of 30-60 mg/kg by intravenous infusion over a first period of time for a first infusion and a second period of time for a second infusion. In some cases, the first period of time is longer than the second period of time. In some cases, the second infusion is the second administration of the antibody. In some cases, the second infusion is the third administration of the antibody. In some cases, the subject receives a dose of 30 mg/kg by intravenous infusion over a period of 30 minutes for the first period of time for the first infusion and a period of 15 minutes for the second period of time for the second infusion. In some cases, the subject receives a dose of 45 mg/kg by intravenous infusion over a period of 30 minutes for the first period of time for the first infusion and a period of 15 minutes for the second period of time for the second infusion. In some cases, the subject receives a dose of 60 mg/kg by intravenous infusion over a period of 60 minutes for the first period of time for the first infusion and a period of 30 minutes for the second period of time for the second infusion. The dosage and rate of administration of the pharmaceutical compositions depends on factors including the prior treatment of the subject, the disease to be treated, and physical characteristics, e.g., age, weight, general health, of the subject.

TABLE 3
Examples of Dosing Regimens

| | | Time for | Time for | Time for any |
|---|---|---|---|---|
| | | first | second | subsequent |
| Dose | Frequency | infusion | infusion | infusion |
| 30 mg/kg | Every two weeks | 30 min | 30 min | 15 min |
| 30 mg/kg | Every two weeks | 30 min | 15 min | 15 min |
| 30 mg/kg | Every two weeks | 15 min | 15 min | 15 min |
| 30 mg/kg | Every four weeks | 30 min | 30 min | 15 min |
| 30 mg/kg | Every four weeks | 30 min | 15 min | 15 min |
| 30 mg/kg | Every four weeks | 15 min | 15 min | 15 min |
| 45 mg/kg | Every two weeks | 30 min | 30 min | 15 min |
| 45 mg/kg | Every two weeks | 30 min | 15 min | 15 min |
| 45 mg/kg | Every two weeks | 15 min | 15 min | 15 min |
| 45 mg/kg | Every four weeks | 30 min | 30 min | 15 min |
| 45 mg/kg | Every four weeks | 30 min | 15 min | 15 min |
| 45 mg/kg | Every four weeks | 15 min | 15 min | 15 min |
| 60 mg/kg | Every two weeks | 60 min | 60 min | 30 min |
| 60 mg/kg | Every two weeks | 60 min | 30 min | 30 min |
| 60 mg/kg | Every two weeks | 30 min | 30 min | 30 min |
| 60 mg/kg | Every four weeks | 60 min | 60 min | 30 min |
| 60 mg/kg | Every four weeks | 60 min | 30 min | 30 min |
| 60 mg/kg | Every four weeks | 30 min | 30 min | 30 min |

TABLE 3
Examples of Additional Dosing Regimens

| | | Time for | Time for | Time for any |
|---|---|---|---|---|
| | | first | second | subsequent |
| Dose | Frequency | infusion | infusion | infusion |
| 30 mg/kg | Twice a month | 30 min | 30 min | 15 min |
| 30 mg/kg | Twice a month | 30 min | 15 min | 15 min |
| 30 mg/kg | Twice a month | 15 min | 15 min | 15 min |
| 30 mg/kg | Twice a month | 30 min | 30 min | 15 min |
| 30 mg/kg | Twice a month | 30 min | 15 min | 15 min |
| 30 mg/kg | Twice a month | 15 min | 15 min | 15 min |
| 45 mg/kg | Twice a month | 30 min | 30 min | 15 min |
| 45 mg/kg | Twice a month | 30 min | 15 min | 15 min |
| 45 mg/kg | Twice a month | 15 min | 15 min | 15 min |
| 45 mg/kg | Twice a month | 30 min | 30 min | 15 min |
| 45 mg/kg | Twice a month | 30 min | 15 min | 15 min |
| 45 mg/kg | Twice a month | 15 min | 15 min | 15 min |
| 60 mg/kg | Twice a month | 60 min | 60 min | 30 min |
| 60 mg/kg | Twice a month | 60 min | 30 min | 30 min |
| 60 mg/kg | Twice a month | 30 min | 30 min | 30 min |
| 60 mg/kg | Once a month | 60 min | 60 min | 30 min |
| 60 mg/kg | Once a month | 60 min | 30 min | 30 min |
| 60 mg/kg | Once a month | 30 min | 30 min | 30 min |

In some embodiments, the anti-FcRn antibodies are administered at the rate disclosed herein without the subject experiencing serious adverse events or reactions.

### VI. Methods of Treatment and Indications

The blockade of human FcRn by anti-FcRn antibodies may be of therapeutic benefit in diseases that are driven by IgG autoantibodies. The ability of FcRn blockade to induce overall IgG catabolism and removal of multiple species of autoantibodies without perturbing serum albumin, small circulating metabolites, or lipoproteins offers a method to expand the utility and accessibility of an autoantibody removal strategy to patients with autoantibody-driven autoimmune disease pathology. While the disclosure is not bound by theory, the dominant mechanism of action of an anti-FcRn antibody may be to increase the catabolism of pathogenic autoantibodies in circulation and decrease autoantibody and immune complex deposition in affected tissues.

The pharmaceutical compositions and methods containing one or more anti-FcRn antibodies (e.g., N022-N024, N026, and N027, preferably N027 and/or N024) are useful to promote catabolism and clearance of pathogenic antibodies, e.g., IgG and IgG autoantibodies in a subject, to reduce the immune response, e.g., to block immune complex-based activation of the immune response in a subject, and to treat immunological conditions or diseases in a subject. In particular, the pharmaceutical compositions and methods are useful to reduce or treat an immune complex-based activation of an acute or chronic immune response. The acute immune response may be activated by a medical condition selected from the group consisting of pemphigus vulgaris, lupus nephritis, myasthenia gravis, Guillain-Barre syndrome, antibody-mediated rejection, catastrophic anti-phospholipid antibody syndrome, immune complex-mediated vasculitis, glomerulitis, a channelopathy, neuromyelitis optica, autoimmune hearing loss, idiopathic thrombocytopenia purpura (ITP), autoimmune haemolytic anaemia (AIHA), immune neutropenia, dialated cardiomyopathy, and serum sickness. The chronic immune response may be activated by a medical condition selected from the group consisting of chronic inflammatory demyelinating polyneuropathy (CIDP), systemic lupus, a chronic form of a disorder indicated for acute treatment, reactive arthropathies, primary biliary cirrhosis, ulcerative colitis, and antineutrophil cytoplasmic antibody (ANCA)-associated vasculitis.

In some embodiments, the pharmaceutical compositions and methods are useful to reduce or treat a disorder selected from the group consisting of alopecia areata, ankylosing spondylitis, antiphospholipid syndrome, Addison's disease, warm autoimmune hemolytic anemia (AIHA), hemolytic anemia, autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, membranous glomerulonephritis, myasthenia gravis, hemolytic disease of the fetus and newborn (HDFN), chronic inflammatory demyelinating polyneuropathy (CIDP), membranous nephropathy, good pasture, polymyositis, Idiopathic thrombocytopenic purpura (ITP; also called “immune thrombocytopenia”), scleroderma, palindromic rheumatism, graves disease, autoimmune thyroiditis, polyglandular autoimmune syndrome, glomerular nephritis, lupus nephritis, systemic lupus erythematosus (SLE), Sjogren's syndrome, Type-1 diabetes, and Wegener's granulomatosis.

In particular, the pharmaceutical compositions and methods are useful to reduce or treat an immune response activated by systemic lupus erythematosus, antiphospholipid syndrome, pemphigus vulgaris/bullous pemphigoid, antineutrophil cytoplasmic antibody (ANCA)-associated vasculitis, myasthenia gravis, or neuromyelitis optica.

In some embodiments, the pharmaceutical compositions and methods are useful to decrease the risk of or decrease the risk of developing anemia in the fetus. In some embodiments, the pharmaceutical compositions and methods are useful to decrease or obviate the need for IUT (intrauterine transfusion). In some embodiments, the pharmaceutical compositions and methods are useful to decrease or obviate the need for antenatal PP+IVIg, postnatal transfusion, IVIg, and/or phototherapy.

In some embodiments, the pharmaceutical compositions and methods are useful to reduce or treat an immune response activated by an autoimmune disease. The autoimmune disease may be selected from the group consisting of alopecia areata, ankylosing spondylitis, antiphospholipid syndrome (e.g., antiphospholipid antibody syndrome), Addison's disease, hemolytic anemia (e.g., warm autoimmune hemolytic anemia), autoimmune hepatitis, hepatitis, Behcets disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, epidermolysis bullosa; fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, membranous nephropathy, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, and Wegener's granulomatosis. In some embodiments, the pharmaceutical compositions and methods are useful to reduce or treat an immune response in a fetus or neonate. In some embodiments, the pharmaceutical compositions and methods are useful to reduce or treat an immune response in a fetus or neonate activated by an autoimmune disease in the pregnant mother.

In particular, the pharmaceutical compositions and methods are useful to reduce or treat an immune response activated by systemic lupus erythematosus, antiphospholipid syndrome, pemphigus vulgaris/bullous pemphigoid, antineutrophil cytoplasmic antibody (ANCA)-associated vasculitis, myasthenia gravis, or neuromyelitis optica. In some embodiments, the pharmaceutical compositions and methods are useful to reduce or treat an immune response in a fetus or neonate. In some embodiments, the pharmaceutical compositions and methods are useful to reduce or treat an immune response activated by systemic lupus erythematosus, antiphospholipid syndrome, pemphigus vulgaris/bullous pemphigoid, antineutrophil cytoplasmic antibody (ANCA)-associated vasculitis, myasthenia gravis, or neuromyelitis optica in the pregnant mother.

The pharmaceutical compositions and methods are useful in methods of decreasing pathogenic antibody transport (e.g., pathogenic maternal IgG antibody transport) across the placenta of a pregnant subject, increasing pathogenic antibody catabolism in a pregnant subject, and treating an antibody-mediated enhancement of viral disease in a fetus or a neonate by administering to a pregnant subject an isolated antibody that binds to human FcRn. Diseases and disorders that may benefit from FcRn inhibition by the isolated anti-FcRn antibodies described herein (e.g., N022-N024, N026, and N027, preferably N027 and/or N024) include diseases and disorders in a fetus and/or neonate that are caused by the transfer of maternal pathogenic antibodies (e.g., maternal pathogenic IgG antibodies) across the placenta from a pregnant subject to the fetus and/or neonate.

In some embodiments, the diseases and disorders that may benefit from FcRn inhibition by the isolated anti-FcRn antibodies described herein (e.g., N022-N024, N026, and N027, preferably N027 and/or N024) are fetal and neonatal alloimmune and/or autoimmune disorders. Fetal and neonatal alloimmune disorders are disorders in a fetus and/or neonate that is caused by pathogenic antibodies in the pregnant subject. The pathogenic antibodies in the pregnant subject may attack the antigens of the fetus (e.g., antigens the fetus inherited from the fetus' father), causing the fetus or the neonate to have a fetal and neonatal alloimmune and/or autoimmune disorder.

Examples of fetal and neonatal alloimmune and/or autoimmune disorders that may be treated by the methods described herein include, but are not limited to, fetal and neonatal alloimmune thrombocytopenia (FNAIT), hemolytic disease of the fetus and newborn (HDFN), alloimmune pan-thrombocytopenia, congenital heart block, fetal arthrogryposis, neonatal myasthenia gravis, neonatal autoimmune hemolytic anemia, neonatal anti-phospholipid syndrome, neonatal polymyositis, dermatomyositis, neonatal lupus, neonatal scleroderma. Behcet's disease, neonatal Graves' disease, neonatal Kawasaki disease, neonatal autoimmune thyroid disease, and neonatal type I diabetes mellitus.

In some embodiments, the diseases and disorders that may benefit from FcRn inhibition by the isolated anti-FcRn antibodies described herein (e.g., N022-N024, N026, and N027, preferably N027 and/or N024) are viral diseases wherein antibodies facilitate viral entry into host cells, leading to increased or enhanced infectivity in the cells, e.g., antibody-mediated enhancement of viral disease. In some embodiments, an antibody may bind to a viral surface protein and the antibody/virus complex may bind to an FcRn on a cell surface through interaction between the antibody and the receptor. Subsequently, the antibody/virus complex may get internalized into the cell. For example, a virus may gain entry into the cells and/or tissues of a fetus through forming a complex with a maternal IgG antibody. A maternal IgG antibody may bind to a viral surface protein and the IgG/virus complex may bind to an FcRn in the syncytiotrophoblasts of the placenta, which then transfers the complex into the fetus.

In some embodiments, the methods described herein may be used to treat an antibody-mediated enhancement of viral disease. In some embodiments, the viral diseases that are enhanced by pathogenic antibodies (e.g., pathogenic IgG antibodies) include, but are not limited to, viral diseases caused by an alpha virus infection, flavivirus infection, Zika virus infection, Chikungunya virus infection, Ross River virus infection, severe acute respiratory syndrome coronavirus infection, Middle East respiratory syndrome, avian influenza infection, influenza virus infection, human respiratory syncytial virus infection, Ebola virus infection, yellow fever virus infection, dengue virus infection, human immunodeficiency virus infection, respiratory syncytial virus infection, Hantavirus infection, Getah virus infection, Sindbis virus infection, Bunyamwera virus infection, West Nile virus infection, Japanese encephalitis virus B infection, rabbitpox virus infection, lactate dehydrogenase elevating virus infection, reovirus infection, rabies virus infection, foot-and-mouth disease virus infection, porcine reproductive and respiratory syndrome virus infection, simian hemorrhagic fever virus infection, equine infectious anemia virus infection, caprine arthritis virus infection, African swine fever virus infection, lentivirus infection, BK papovavirus infection, Murray Valley encephalitis virus infection, enterovirus infection, cytomegalovirus infection, pneumovirus infection, morbillivirus infection, and measles virus infection.

The blockade of human FcRn by anti-FcRn antibodies may be of therapeutic benefit in diseases that are driven by pathogenic antibodies (e.g., pathogenic IgG antibodies). The ability of FcRn blockade to induce overall pathogenic antibody catabolism and removal of multiple species of pathogenic antibodies, small circulating metabolites, or lipoproteins offers a method to expand the utility and accessibility of a pathogenic antibody removal strategy to patients with pathogenic antibody-driven autoimmune disease pathology. While not bound by theory, the dominant mechanism of action of an anti-FcRn antibody may be to increase the catabolism of pathogenic antibodies in circulation and decrease pathogenic antibody and immune complex deposition in affected tissues.

The anti-FcRn antibodies described herein (e.g., N022-N024, N026, and N027, preferably N027 and/or N024) may be administered to a pregnant subject who has or is at risk of having a medical condition that activates an immune response in the pregnant subject. In some embodiments, the pregnant subject may have had, in the past, a medical condition that activated an immune response in the pregnant subject. In some embodiments, the pregnant subject has a history of having had a previous fetus or neonate that had a fetal and neonatal alloimmune and/or autoimmune disorder. In some embodiments, the anti-FcRn antibodies described herein may be administered to a pregnant subject if a pathogenic antibody associated with an immune disease is detected in a biological sample (e.g., a blood or urine sample) obtained from the pregnant subject. In some embodiments, the pathogenic antibody detected in the biological sample of the pregnant subject is known to bind to an antigen from the fetus in the pregnant subject (e.g., an antigen that the fetus inherited from the fetus' father).

In some embodiments, the anti-FcRn antibodies described herein (e.g., N022-N024, N026, and N027, preferably N027 and/or N024) may be administered to a subject who is planning to become pregnant and who has or is at risk of having a medical condition that activates an immune response in the pregnant subject, and/or who has had, in the past, a medical condition that activated an immune response in the pregnant subject. In some embodiments, a subject is planning to become pregnant and has a history of having had a previous fetus or neonate that had a fetal and neonatal alloimmune and/or autoimmune disorder. In some embodiments, the anti-FcRn antibodies described herein may be administered to a subject who is planning to become pregnant and whose biological sample contains a pathogenic antibody associated with an immune disease.

In some embodiments, the anti-FcRn antibodies described herein may be administered to a subject (e.g., a pregnant subject) to reduce or treat an immune complex-based activation of an acute or chronic immune response in the subject. The acute immune response may be activated by a medical condition (e.g., pemphigus vulgaris, lupus nephritis, myasthenia gravis, Guillain-Barre syndrome, antibody-mediated rejection, catastrophic anti-phospholipid antibody syndrome, immune complex-mediated vasculitis, glomerulitis, a channelopathy, neuromyelitis optica, autoimmune hearing loss, idiopathic thrombocytopenia purpura, autoimmune haemolytic anaemia, immune neutropenia, dialated cardiomyopathy, serum sickness, chronic inflammatory demyelinating polyneuropathy, systemic lupus, reactive arthropathies, primary biliary cirrhosis, ulcerative colitis, or antineutrophil cytoplasmic antibody (ANCA)-associated vasculitis).

In some embodiments, the anti-FcRn antibodies described herein may be administered to a subject (e.g., a pregnant subject) to reduce or treat an immune response activated by an autoimmune disease. The autoimmune disease may be, for example, alopecia areata, ankylosing spondylitis, antiphospholipid syndrome, Addison's disease, hemolytic anemia, warm autoimmune hemolytic anemia (wAIHA), anti-factor antibodies, heparin induced thrombocytopenia (HICT), sensitized transplant, autoimmune hepatitis, hepatitis, Behcet's disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatricial pemphigoid, limited scleroderma (CREST syndrome), cold agglutinin disease, Crohn's disease, dermatomyositis, discoid lupus, essential mixed cryoglobulinemia, fibromyalgia, fibromyositis, Graves' disease, Hashimoto's thyroiditis, hypothyroidism, inflammatory bowel disease, autoimmune lymphoproliferative syndrome, idiopathic pulmonary fibrosis, IgA nephropathy, insulin dependent diabetes, juvenile arthritis, lichen planus, lupus, Meniere's Disease, mixed connective tissue disease, multiple sclerosis, pernicious anemia, polyarteritis nodosa, polychondritis, polyglandular syndromes, polymyalgia rheumatica, polymyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis, ulcerative colitis, uveitis, vitiligo, or Wegener's granulomatosis.

### EXAMPLES

The Various FcRn Antibodies Described Herein and their Properties are Described in Detail in WO 2019/118791 (PCT/US2018/065568).

### Example 1—IgG Competition

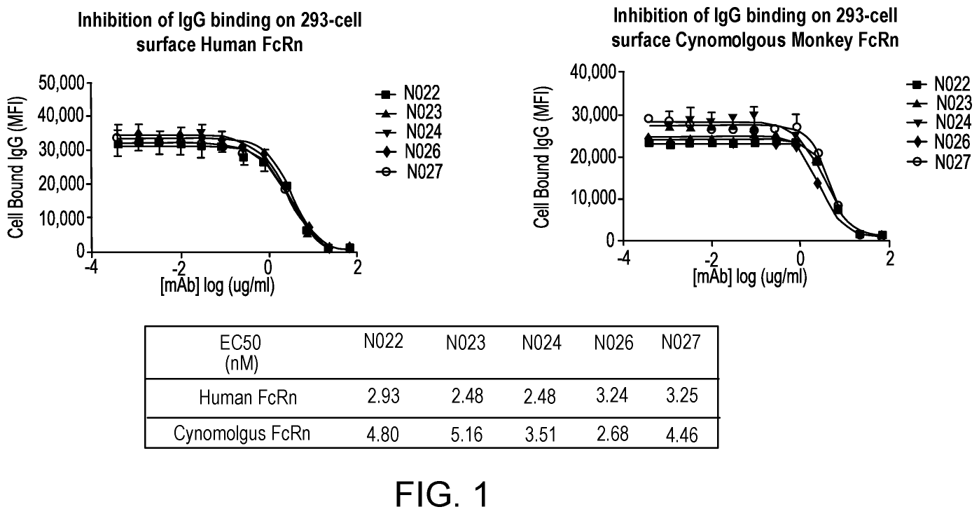

The ability of anti-FcRn antibodies to compete with IgG for binding to human or cynomolgus monkey FcRn was evaluated on human embryonic kidney (HEK) 293 cells ectopically expressing cell surface, glycophosphatidylinositol (GPI)-linked FcRn. Human and cynomolgus monkey FcRn alpha amino acid sequences exhibit 97.5% sequence identity. Nine amino acid residues of 355 are different between human and cynomolgus monkey FcRn alpha, but none are in the epitope-mapped binding region. The level of cell-bound IgG was determined using 66 nM of fluorescent probe-labeled, non-specific IgG. The binding of IgG to cell surface FcRn was done at pH 6.0, which allows the Fc portion of IgG to interact with FcRn. As shown in FIG. 1, the amount of cell-bound IgG significantly decreased as the concentration of the anti-FcRn antibody (N022-N024, N026, or N027) increased. The binding of IgG was inhibited in a concentration- and saturation-dependent manner by each of the five exemplary anti-FcRn antibodies of the disclosure, demonstrating the ability of the anti-FcRn antibodies, N022-N024, N026, and N027, to effectively compete with and inhibit binding of IgG to FcRn at pH 6.0. The EC50 values of the antibodies ranged between 2 and 6 nM.

### Example 2—Effect of Anti-FcRn Antibodies on IgG Catabolism in Mice

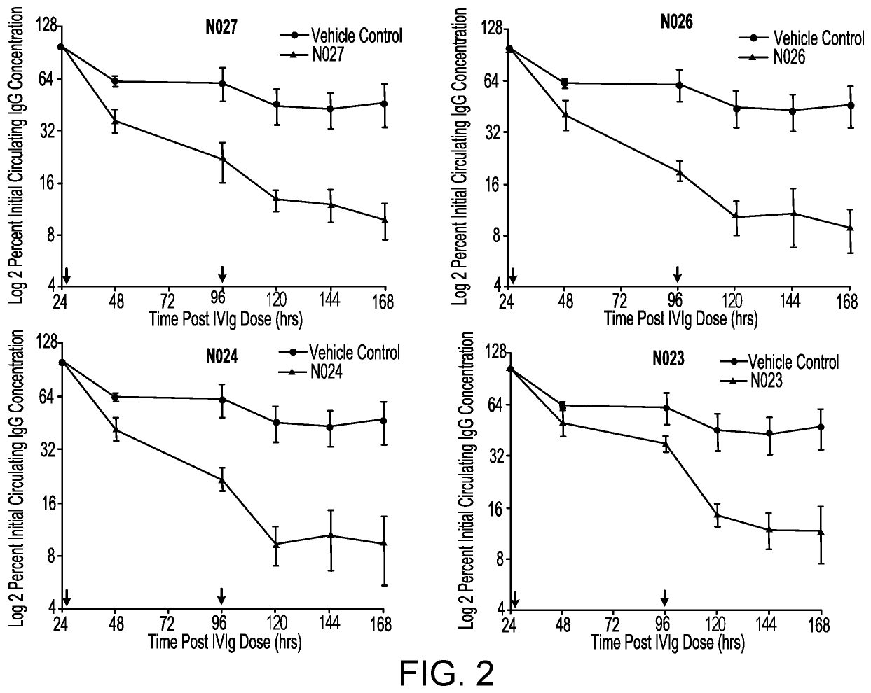

To measure the effect of the anti-FcRn antibodies on IgG catabolism in vivo, human FcRn transgenic mouse strain FcRn−/−hFcRn (32) Tg mice, which lacks mouse FcRn but expresses human FcRn in a tissue distribution similar to the endogenous mouse and human FcRn, was used. FcRn−/−hFcRn (32) Tg mice injected with 500 mg/kg human IgG on day 0 were administered a single dose of an anti-FcRn antibody at 10 mg/kg on days 1 and 4. As shown in FIG. 2, the catabolism of IgG was increased by the administration of anti-FcRn antibodies as seen by lower levels of IgG measured over time in anti-FcRn antibody-treated mice. The activities of N024 (K_{D}=35.5 pM), N026 (K_{D}=36.5 pM), and N027 (K_{D}=19.4 pM) appeared to be to be similar at 10 mg/kg.

### Example 3—In Vitro and In Vivo Functional Characterizations of Anti-FcRn Antibodies

In Vitro

Cellular binding affinities of the antibodies were measured on human embryonic kidney (HEK) 293 cells ectopically expressing cell surface, glycophosphatidylinositol (GPI)-linked human or cynomolgus monkey FcRn. FcRn is a type I transmembrane protein with the IgG and albumin binding domains oriented to the luminal side of endosomal membranes or to the cell surface when transported to the plasma membrane. The binding of anti-FcRn antibodies to cell surface, membrane-associated FcRn on HEK293 cells at pH 7.4 mimics binding in a physiologically-relevant environment and at the pH where only the Fab domain and not the Fc domain of the antibodies interact with FcRn. The FcRn extracellular domain was displayed on the cell surface at high density through a C-terminal engineered GPI linkage. The anti-FcRn antibodies were labeled with a fluorescent probe. The antibodies were allowed to bind for 30 minutes on ice. Cells were then washed at 4° C. and bound antibodies were detected using a fluorophore-labeled secondary antibody, e.g., a goat anti-human IgG F(ab)₂. The binding to human FcRn was concentration dependent and antibodies displayed EC50 values ranging from 4 to 7 nM.

Cellular binding affinities of the antibodies were also measured on endogenously expressed human FcRn. Monocytes express the highest levels of FcRn and show the highest percent positivity for FcRn expression in mouse and human blood. Monocytic cell line THP-1 was used to evaluate binding of anti-FcRn antibodies to endogenous human FcRn at pH 7.4. Since endogenous FcRn is primarily in intracellular endosomal vesicles in THP-1 cells, the cells were first permeablized with a mild detergent and fixed prior to incubation for 30 minutes at 4° C. with anti-FcRn antibodies in the presence of bovine serum to block non-specific Fc receptor binding. This assay was able to distinguish antibodies with better binding to endogenous human FcRn. The binding of anti-FcRn antibodies to THP-1 cells is concentration dependent. All antibodies of the disclosure, e.g., N022-N024, N026, and N027, showed better binding affinities than IgG1. Antibody N027 displayed the highest binding affinity with an EC50 value of 3.0 nM.

The ability of anti-FcRn antibodies to compete with IgG for binding to human or cynomolgus monkey FcRn was evaluated on human embryonic kidney (HEK) 293 cells ectopically expressing cell surface, GPI-linked FcRn. The level of cell-bound IgG was determined using fluorescent probe-labeled, non-specific IgG. The binding of IgG to cell surface FcRn was done at pH 6.0, which allows the Fc portion of IgG to interact with FcRn. As shown in Example 3 and FIG. 1, the amount of cell-bound IgG significantly decreased as the concentration of the anti-FcRn antibody increased. The binding of IgG was inhibited in a concentration- and saturation-dependent manner by each of the five exemplary anti-FcRn antibodies of the disclosure, e.g., N022-N024, N026, and N027, demonstrating the ability of the anti-FcRn antibodies to effectively compete with and inhibit binding of IgG to FcRn at pH 6.0. The EC50 values of the antibodies ranged from 2 to 6 nM.

Epitope mapping by hydrogen-deuterium exchange of the antibodies indicated that the antibodies bind to an epitope on human FcRn located in and/or adjacent to the Fc-FcRn interaction interface, which suggests that the antibodies block IgG binding to FcRn by direction inhibition. Furthermore, the epitope-mapped binding site is distant from the albumin-binding site of FcRn. An enzyme-linked immunosorbent assay (ELISA) was used to confirm that the antibodies do not inhibit serum albumin binding to FcRn. Soluble His-tagged extracellular domain of human FcRn was bound to the plate surface and pre-incubated with increasing concentrations of anti-FcRn antibody at pH 6.0. Horseradish peroxidase (HRP)-conjugated human serum albumin was allowed to bind to the soluble, His-tagged FcRn. None of the antibodies inhibited albumin binding to FcRn. Furthermore, in vivo experimental evidence also showed that mouse albumin levels remained constant after anti-FcRn antibody administration, indicating that albumin recycling was not disturbed by antibody binding to FcRn.

In Vivo

To test the in vivo effect of anti-FcRn antibodies on IgG catabolism, human FcRn transgenic mouse strain FcRn−/−hFcRn (32) Tg mice, which lack mouse FcRn but express human FcRn in a tissue distribution similar to that of the endogenous mouse and human FcRn, were used. FcRn−/−hFcRn (32) Tg mice injected with human IgG on day 0 were administered a single dose of an anti-FcRn antibody at 10 mg/kg on days 1 and 4. As shown in FIG. 2, the catabolism of IgG was increased by the administration of anti-FcRn antibodies as seen by lower levels of IgG measured over time in anti-FcRn antibody-treated mice. The activities of N024 (K_{D}=35.5 pM), N026 (K_{D}=36.5 pM), and N027 (K_{D}=19.4 pM) appeared to be to be similar at 10 mg/kg.

### Example 4—Effect of Anti-FcRn Antibodies on IgG Levels and Target Occupancy in Mice

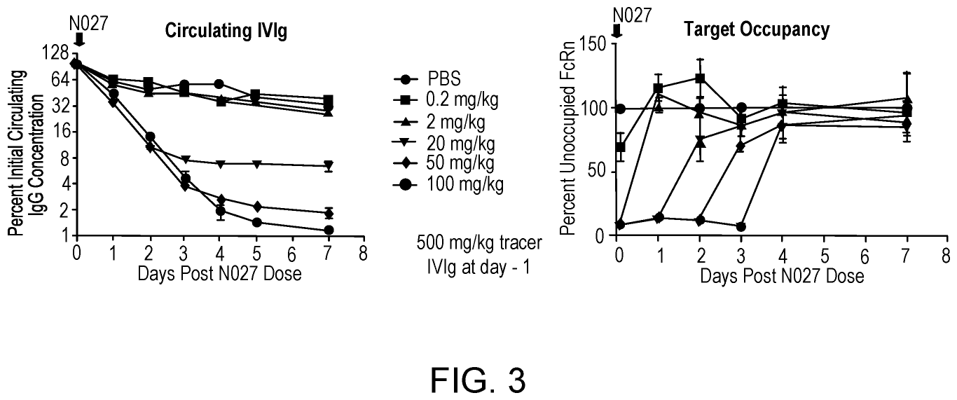

N027 was dosed intravenously (i.v.) 24 hrs after administration of 500 mg/kg IVIg (tracer) to Tg32 human FcRn (hFCGRT) transgenic, mouse FcRn (mFCGRT) knockout mice. Circulating human IgG was detected by ELISA on each day. Target occupancy was measured on each day in monocytes from lysed whole blood by fluorescence-activated cell sorting (FACS), after incubation of cells with immunophenotyping cell surface markers followed by fixation and permeabilization. Unoccupied FcRn was measured by staining with Dy650-labeled N027 (n=4 males per group). As shown in FIG. 3, IgG level and the percentage of unoccupied FcRn were decreased by the administration of N027 in a dose-dependent manner.

### Example 5—Selective Induction of IgG Catabolism and Target Occupancy in Cynomolgus Monkeys

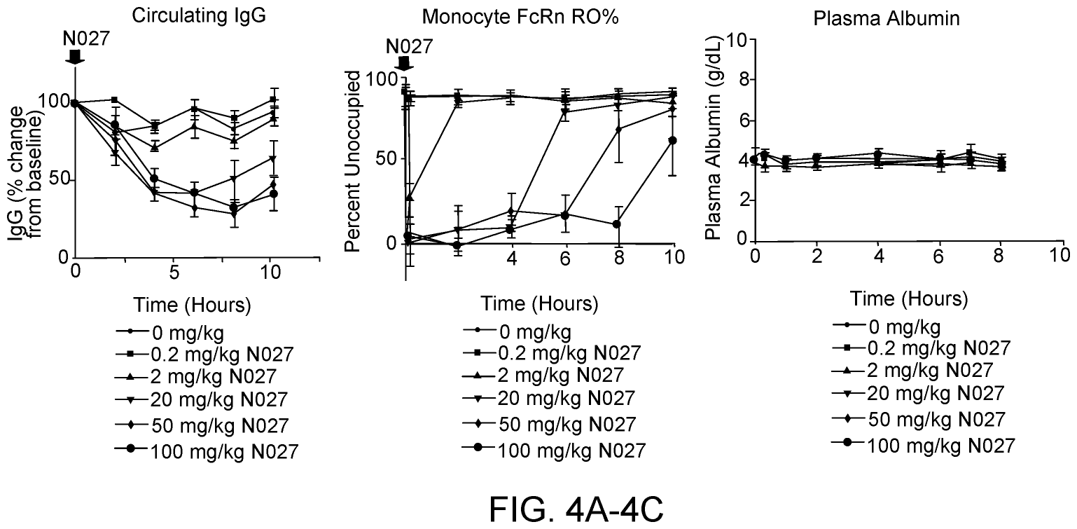

N027 was dosed i.v. at t=0 in cynomolgus monkeys. Circulating endogenous IgG and albumin was detected by ELISA. Target occupancy was measured in monocytes from lysed whole blood by FACS, after incubation of cells with immunophenotyping cell surface markers followed by fixation and permeabilization. Unoccupied FcRn was measured by staining with Dy650-labeled N027. (n=3 males per group). As shown in FIG. 4, IgG level and the percentage of unoccupied FcRn were decreased by the administration of N027 in a dose-dependent manner, while plasma albumin level stayed unchanged.

### Example 6—Efficacy of N027 in Mouse Chronic Idiopathic Thrombocytopenia Purpura (ITP)

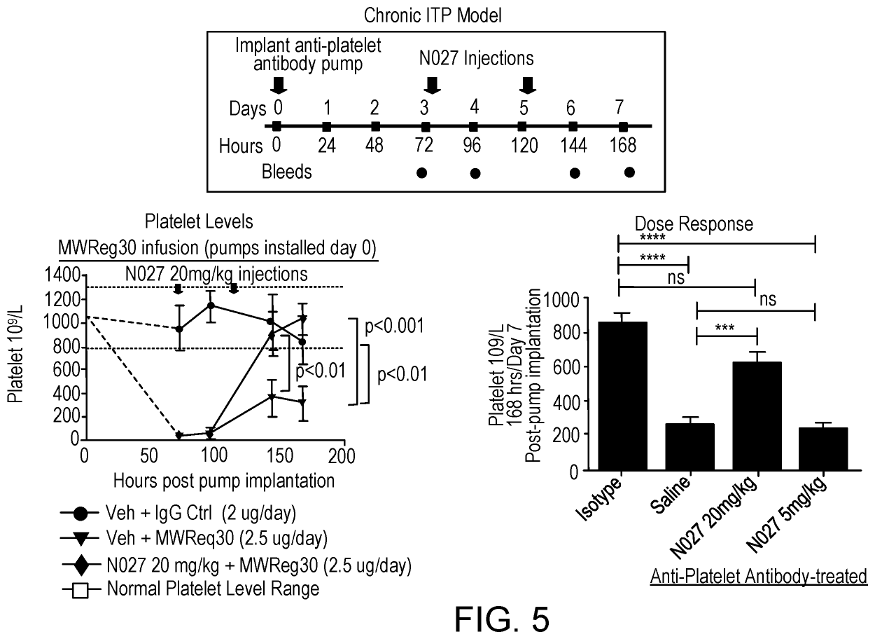

Thrombocytopenia was induced in Tg32 human FcRn (hFCGRT) transgenic, mouse FcRn (mFCGRT) knockout mice by continuous infusion of anti-platelet antibody (anti-CD41, MWReg30) subcutaneous (s.c.) miniosmotic pump. Circulating platelet levels were decreased to 300×109/L or less by 72 hrs (Day 3) after pump implantation. N027 was dosed therapeutically i.v. 72 hrs (day 3) and 120 hrs (Day 5) post-pump implantation (A, n=4 per group; B, n=7 per group). FIG. 5 shows the effects of N027 on platelet levels in mice having thrombocytopenia.

### Example 7—Safety and Tolerability of Intravenous Infusion of an Anti-FcRn Antibody

A single-dose, sequential, randomized, double-blind (Sponsor-open), placebo-controlled, escalating dose and escalating infusion rate study of an antibody having the light chain of SEQ ID NO:19 and the heavy chain sequence of SEQ ID NO:24 (N027; M281) was conducted. Subjects were randomized to receive a single dose of 30 or 60 mg/kg antibody or placebo by intravenous infusion on Day 1. Each of five cohorts consisted of six subjects receiving antibody and two subjects receiving placebo for a total of 40 subjects. The five cohorts were: 30 mg/kg antibody administered over 60 minutes (6 subjects) or placebo (2 subjects); 30 mg/kg antibody administered over 30 minutes (6 subjects) or placebo (2 subjects); 30 mg/kg antibody administered over 15 minutes (6 subjects) or placebo (2 subjects); 30 mg/kg antibody administered over 7.5 minutes (6 subjects) or _{placebo (2} subjects); and 60 mg/kg antibody administered over 15 minutes (6 subjects) or placebo (2 subjects). The concentration of the antibody in the intravenous infusion was 30 mg/ml.

There were no deaths, serious adverse events (SAEs) or adverse events leading to subject withdrawal from the study. The most commonly reported treatment emergent adverse events were: headache, reported by 6 (20%) subjects in the active treatment groups and 1 (10%) subject receiving placebo and nausea, reported by 3 (10%) subjects receiving active treatment. Both 30 mg/Kg infused in 7.5 min and 60 mg/Kg infused in 15 min, although tolerated appeared to have higher rates of headache and nausea than at lower infusion rates.

### Other Embodiments

While the disclosure has been described in connection with specific embodiments thereof, it will be understood that it is capable of further modifications and this application is intended to cover any variations, uses, or adaptations following, in general, the principles and including such departures from the present disclosure come within known or customary practice within the art to which the disclosure pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

Other embodiments are within the following claims.

1.-88. (canceled)

89. A method of treating a subject with an alloimmune and/or autoimmune disorder, the method comprising intravenously administering a pharmaceutical composition comprising an anti-FcRn antibody at a dose of about 5 mg/kg to about 60 mg/kg of the anti-FcRn antibody to the subject in about 45 minutes or less,
wherein the anti-FcRn antibody comprises: (1) a light chain variable region comprising a CDR L1, a CDR L2, and a CDR L3 and (2) a heavy chain variable region comprising a CDR H1, a CDR H2, and a CDR H3, wherein
the CDR L1 comprises a sequence having no more than two amino acid substitutions relative to the sequence of TGTGSDVGSYNLVS (SEQ ID NO: 1),
the CDR L2 comprises a sequence having no more than one amino acid substitutions relative to the sequence of GDSERPS (SEQ ID NO: 2),
the CDR L3 comprises a sequence having no more than one amino acid substitutions relative to the sequence of SSYAGSGIYV (SEQ ID NO: 3),
the CDR H1 comprises a sequence having no more than one amino acid substitutions relative to the sequence of TYAMG (SEQ ID NO: 4), DYAMG (SEQ ID NO: 5), or NYAMG (SEQ ID NO: 6),
the CDR H2 comprises a sequence having no more than two amino acid substitutions relative to the sequence of SIGSSGAQTRYADS (SEQ ID NO: 7), SIGASGSQTRYADS (SEQ ID NO: 8), SIGASGAQTRYADS (SEQ ID NO: 9), or SIGASGGQTRYADS (SEQ ID NO: 10), and
the CDR H3 comprises a sequence having no more than one amino acid substitutions relative to the sequence of LAIGDSY (SEQ ID NO: 11).

90. The method of claim 89, wherein
the CDR L1 comprises the sequence TGTGSDVGSYNLVS (SEQ ID NO: 1),
the CDR L2 comprises the sequence GDSERPS (SEQ ID NO: 2),
the CDR L3 comprises the sequence SSYAGSGIYV (SEQ ID NO: 3),
the CDR H1 comprises the sequence TYAMG (SEQ ID NO: 4),
the CDR H2 comprises the sequence SIGASGSQTRYADS (SEQ ID NO: 8), and
the CDR H3 comprises the sequence LAIGDSY (SEQ ID NO: 11).

91. The method of claim 89, wherein the antibody is administered to the subject in about 7-45 minutes, about 7-30 minutes, about 10-45 minutes, about 10-30 minutes, or about 15-30 minutes.

92. The method of claim 89, wherein a Fc domain of the antibody is not glycosylated.

93. The method of claim 89, wherein the subject has a alloimmune and/or autoimmune disorder selected from the group consisting of fetal and neonatal alloimmune thrombocytopenia, hemolytic disease of the fetus and newborn, alloimmune pan-thrombocytopenia, congenital heart block, fetal arthrogryposis, neonatal myasthenia gravis, neonatal autoimmune hemolytic anemia, neonatal anti-phospholipid syndrome, neonatal polymyositis, dermatomyositis, neonatal lupus, neonatal scleroderma, Behcet's disease, neonatal Graves' disease, neonatal Kawasaki disease, neonatal autoimmune thyroid disease, and neonatal type I diabetes mellitus.

94. The method of claim 89, wherein the subject has a alloimmune and/or autoimmune disorder is selected from the group consisting of thrombocytopenia, pan-thrombocytopenia, congenital heart block, arthrogryposis, myasthenia gravis, autoimmune hemolytic anemia, warm autoimmune hemolytic anemia, anti-phospholipid syndrome, polymyositis, dermatomyositis, lupus, scleroderma, Behcet's disease, Graves' disease, Kawasaki disease, autoimmune thyroid disease, and type I diabetes mellitus.

95. The method of claim 89, wherein the pharmaceutical composition comprises the anti-FcRn antibody at concentration of about 15 mg/ml, about 30 mg/ml, about 45 mg/ml, or about 60 mg/ml.

96. The method of claim 89, wherein the pharmaceutical composition comprises:
i) the antibody at a concentration of 10-60 mg/ml;
ii) 20-30 mM sodium phosphate;
iii) 20-30 mM sodium chloride;
iv) 80-100 mg/ml Trehalose; and
v) 0.1-0.005% w/v Polysorbate 80.

97. The method of claim 89, wherein the light chain comprises a sequence of SEQ ID NO: 19 and the heavy chain comprises a sequence of SEQ ID NO:24.

98. The method of claim 89, wherein the dose is about 30 mg/kg

99. The method of claim 89, wherein the dose is about 15 mg/kg.

100. The method of claim 89, wherein the dose is about 45 mg/kg.

101. The method of claim 89, wherein the subject is a pregnant woman.

102. The method of claim 89, wherein the composition is administered at least every other week, every other week, at least every week, or every week.

103. The method of claim 89, wherein the intravenous administration occurs two or more times.

104. The method of claim 103, wherein a first infusion is administered to the subject in about 45 minutes or less, 30 minutes or less, or 15 minutes or less.

105. The method of claim 104, wherein a second fusion and a third fusion are administered to the subject in an amount of time which is reduced compared to the first infusion.

106. The method of claim 89, wherein the dose is about 30 mg/kg and is administered in about 30 minutes.

107. The method of claim 89, wherein the dose is about 30 mg/kg antibody and is administered in about 15 minutes.

108. The method of claim 89, wherein the dose is about 30 mg/kg antibody and is administered in about 7.5 minutes.
